(19) United States
(12) Patent Application Publication
(10) Pub. No.: US 2022/0354852 A1
(43) Pub. Date: Nov. 10, 2022

(54) TARGETING WNT SIGNALING FOR IMPROVED GLIOMA IMMUNOTHERAPY

(71) Applicant: CITY OF HOPE, Duarte, CA (US)

(72) Inventors: Margarita GUTOVA, Duarte, CA (US); Michael KAHN, Duarte, CA (US); Christine BROWN, Duarte, CA (US)

(73) Assignee: CITY OF HOPE, Duarte, CA (US)

(21) Appl. No.: 17/662,629

(22) Filed: May 9, 2022

Related U.S. Application Data
(60) Provisional application No. 63/185,590, filed on May 7, 2021.

(51) Int. Cl. A61K 31/519; A61P 35/00; A61K 35/17; A61K 38/17; A61K 31/675

(52) U.S. Cl. CPC A61K 31/519; A61P 35/00; A61K 35/17; A61K 38/1774; A61K 31/675

(57) ABSTRACT

This disclosure relates to cells that express a chimeric antigen receptor that targets IL13Rα2 and/or HER2 and methods for treating a brain tumor by administering a population of cells expressing a chimeric antigen receptor that targets IL13Rα2 and/or HER2 in combination with a Wnt/β-catenin pathway modulator to a subject having a brain tumor.

### CROSS-REFERENCE TO RELATED APPLICATION

This application claims the benefit of U.S. Patent Application No. 63/185,590, filed May 7, 2021, the disclosure of which is hereby incorporated by reference in its entirety as if fully set forth herein, including all references and appendices submitted therewith.

### BACKGROUND

Brain tumors in children and adults brain tumors [BTs], including ependymoma [EP], medulloblastoma [MB] and glioblastoma [GBM]) are devastating diseases, causing substantial morbidity and mortality (Shergalis, Bankhead et al. 2018, Kabir, Kunos et al. 2020, Khasraw, Reardon et al. 2020). The median survival for BTs ranges from 13 to 73 months with a 5-year survival of less than 20% (Zhang et al. 2013; Song et al. 2010).

Despite aggressive standard-of-care therapy, including surgery, radiation, and chemotherapy, tumor recurrence is almost inevitable and uniformly lethal. Major obstacles to successful treatment of brain tumors include the blood-brain barrier (BBB), which prevents the majority of anti-cancer agents from entering the central nervous system (CNS), and chemotherapy dose limitations because of toxicity to normal brain and other tissues (Muldoon, Soussain et al. 2007). The refractory nature of BTs provides a compelling motivation for developing novel treatment interventions, such as CAR T cell therapy, for these devastating diseases (Garvin, Selch et al. 2012, Zhao, Liu et al. 2012, Brown, Badie et al. 2015, Brown, Alizadeh et al. 2016, Khasraw, Reardon et al. 2020). Encouraging clinical success demonstrating that CD19-CAR T-cells mediate high clinical response rates in patients with refractory B cell malignancies (Zhang, Jiang et al. 2020) provide the foundation for applying this promising therapy to other cancers (Sommer, Boldajipour et al. 2019, Marotte, Simon et al. 2020, Zah, Nam et al. 2020). In support of CAR T-cells as a strategy for brain tumors, CD19-CAR T-cells efficiently traffic to the central nervous system (CNS) in patients and can eliminate metastatic disease in the brain (Brown, Vishwanath et al. 2007, Brown, Alizadeh et al. 2016). However, bioactivity of the therapeutic CAR T cells is only observed in a subset of patients, and therapeutic resistance and relapse pose a significant problem. Thus, improved therapies are needed to address the need to fight these deadly diseases.

### BRIEF DESCRIPTION OF THE DRAWINGS

This application contains at least one drawing executed in color. Copies of this application with color drawing(s) will be provided by the Office upon request and payment of the necessary fees.

FIG. 1. Clinical response and recurrence after CAR T therapy is associated with Wnt pathway upregulation and antigen loss. (A) Regression of recurrent multifocal GBM, including spinal metastases, in a patient (UPN109) after intratumoral (ICT) and intraventricular (ICV) delivery of IL13Rα2-targeted CART cells. (B) Gene expression profile of the tumor before resection and CAR T therapy (Pre-Op) and after therapy (Post CAR T; ICV and ICT). Differentially expressed transcripts/genes from the WNT pathway were seen in the relapsed patient tumor. (C) IHC analysis of Pre-Op and Post CAR T-treated tumor for expression of IL13Rα2, HER2, and EGFR antigens.

FIG. 2. Combinational strategy of CAR T cell-mediated tumor-antigen targeting with ICG-001 induced differentiation in glioma. (A) In vitro differentiation and cell growth arrest of PBT017 glioma cells treated with ICG-001 (0.75-10 μM). (B) Cytotoxicity of HER2 CAR-T cells and tumor cells (1:5 ratio) was enhanced by ICG-001 at concentrations 1.25-5 μM. (C) Treatment of PBT138 patient glioma tumors with CAR T IL13Rα2+/−ICG-001.

FIG. 3. ICG-001 increased expression of interferon-y regulated chemokines. (A) qRT-PCR analysis showing ICG-001 significantly increased the expression of the interferon-y inducible chemokines CXCL9-CXCL1. (B) Increased CXCL9-CXCL11 expression correlated with increased CD8+ T cell infiltration into the tumor microenvironment as judged by IHC (minimally 10 fields counted).

FIG. 4. CBP/β-catenin antagonists force the differentiation of tumor cells thereby decreasing tumor cell glycolytic activity and acidification of the tumor.

FIG. 5. (A) TCGA database analysis of WISP1 expression in LGG and GBM. (B) Survival data of patients with brain tumors expressing high and low levels of Wnt/β catenin target gene WISP 1.

FIG. 6. TCGA data plots demonstrating correlation of expression of WISP1 gene and IL13Rα2, B7-H3 and PDL-1.

FIG. 7. Viability and cell count of PBT glioma cells treated with ICG 001 (0, 4 and 8 μM) for 7 days.

FIG. 8. NanoString analysis of human metabolic pathways after treatment with ICG-001. The profiling panel consists of 770 genes targeting six fundamental themes: neurotransmission, neuron-glia interactions, neuroplasticity, cell structure integrity, neuroinflammation, and metabolism. Each assay also includes six positive and eight negative RNA assay controls, plus ten mRNA housekeeping controls. RNA was hybridized with the Codeset from the gene panel at 65° C. for 16 h. The post-hybridization probe-target mixture were quantified using the nCounter Digital Analyzer, and all data analysis was performed on nSolver (NanoString Technologies) metabolic panel. All raw data was normalized with internal positive and negative controls to eliminate variability unrelated to the samples, then normalized to the selected housekeeping genes using Geometric Means methods.

FIG. 9 shows the design of in vivo studies using PBT 138 cell line orthotopic models.

FIG. 10. Immunohistochemistry staining for CD8+ cell within glioma. (A) An example of a poor CD8+ T cell infiltrated PDX glioma. (B) An example of a highly CD8⁺ T cell infiltrated tumor.

FIG. 11. (A, B) Growth kinetics of human PBT glioma lines (PBT017, PBT030, PBT135, PBT144, and PBT147) treated with ICG-001 (0-10 μM) for 7 days and (C) mouse glioma cells (GL261, GL261-IL13, and K-luc) treated with ICG-001 (0-10 μM) for 4 days. Red boxes indicate PBT030 and PBT147 cell lines used for Nanostring and proteomics analysis. Scale bars represent S.D. of triplicate samples. (D-F) Growth kinetics of PBT017 (2,500 cells/well) treated with or without HER2-CAR T cells (500 cells/well) and with or without ICG-001 (0-5 μM). The red line is CAR T cell treatment only and the bottom three lines are with ICG-001. Experiment was done in quadruplicate and error bars indicate S.D.

FIG. 12 shows bar graphs (A, B) and Images of PBT017 glioma treated with ICG-001 (C-E) as indicated according to some embodiments.

FIG. 13 illustrates data resulting from the combination of ICG001 and CAR T cells toxicity in vitro using 2000 cells to start and 4 days (96-well Endpoint—cell count) according to certain embodiments.

FIG. 14 illustrates data resulting from the Combination of ICG001 and CAR T cells toxicity in vitro using 1000 cells to start and 4 days (96-well Endpoint—cell count) according to certain embodiments.

FIG. 15. Expression of target genes post-ICG-001 treatment (0-10 μM) by RT-PCR using (A-C) mouse Survivin in GL261-IL13, GL261, and K-luc cells. (D) Survivin in PBT147 human glioma cells. (E, F) Luciferase reporter assay indicating downregulation of Survivin/BIRC5 in PBT017 and PBT030 cell lines.

FIG. 16 is a table showing the expression of the target genes survivin/BIRC5 post-ICG-001 treatment (0, 5, 10 mM) as detected by RT-PCR and Luciferase Reporter Assay.

FIG. 17. (A-F) NanoString analysis of human metabolic pathways after treatment with ICG-001. The profiling panel consisted of 770 genes targeting six fundamental themes: neurotransmission, neuron-glia interactions, neuroplasticity, cell structure integrity, neuroinflammation, and metabolism. Each assay also included six positive and eight negative RNA assay controls and ten mRNA housekeeping controls. RNA was hybridized with the Codeset from the gene panel at 65° C. for 16 h. The post-hybridization probe-target mixture was quantified using the nCounter Digital Analyzer and all data analysis was performed using the nSolver metabolic panel (NanoString Technologies). All raw data was normalized with internal positive and negative controls to eliminate variability unrelated to the samples and then normalized to the selected housekeeping genes using Geometric Means methods. PBT147 and PBT030 cells were treated with ICG-001 at 0.5 and 10 μM for 24 h (data not shown) or 72 h. Total RNA was collected for NanoString analysis from triplicate samples.

FIG. 18. Proteomics analysis of PBT147 and PBT030 cell lines treated with ICG-001 (0-10 μM) for 72 h. (A) Protein density was calculated by the difference in log normalized levels for each protein between the four treated samples and the appropriate untreated sample. (B) Calculating the difference in log levels corresponds also to looking at the log of the fold changes in each protein. For both cell lines, the log fold-change for each protein at 5 μg was plotted against the log fold-change at 10 μg. These plots are shown in A, with the points color-coded by cell line. This highlights a positive linear relationship between the log fold changes at 5 μg and 10 μg in both cell lines. The dose effect is significantly greater in PBT030 than PBT147, with the same increase in dose, resulting in a generally greater fold-change. (C) Patterns of similarities in protein regulation over cell lines and/or doses are shown in a heat map of up- or downregulated proteins. The heatmap has been generated using Ward's method for clustering and the Manhattan (L1) distance. To highlight patterns of similar changes in regulation over cell lines and doses, rather than absolute differences in protein levels, quantile normalization has been used before creating the heatmap, with protein levels in each sample being normalized to that of a uniform distribution on −1≤x≤1.

FIG. 19 is a table showing a proteomics analysis at 72 hours for two cell lines (PB147, PB030) treated with ICG-001 in vitro at three doses (0 μg, 5 μg, 10 μg). 1557 proteins were quantified. The table shows a summary of the range of protein levels of the 1557 proteins that were measured in each sample at 72 hours. To account for different number of cells in each sample, and to make numbers comparable across experiments, the levels were normalized by dividing by the measured level for each protein by the total level of protein found in each sample.

FIG. 20 is a table showing the top 20 (relatively) upregulated proteins as a function of the density of change in normalized protein levels following ICG001 treatment in vitro. Proteins shown in bold occur simultaneously on respective cell lines for the 5 μg and 10 μg list. Underlined proteins occur simultaneously on respective both 10 μg lists.

FIG. 21 is a table showing the top 20 (relatively) downregulated proteins as a function of the density of change in normalized protein levels following ICG001 treatment in vitro. Proteins shown in bold occur simultaneously on respective cell lines for the 5 μg and 10 μg list. Underlined proteins occur simultaneously on respective both 10 μg lists.

FIG. 22. IHC analysis of immune cells in syngeneic models of subcutaneous glioma (K-Luc). (A-D) Mice bearing subcutaneous K-luc glioma tumors were treated with ICG-001 (50 mg/kg/day) for 7, 14, or 21 days. At the end of treatment, tumor was excised and IHC stained for CD3 cells to evaluate recruitment and patterns of CD3 distribution (red). (E) Quantification of tumor coverage using ImageJ for mouse CD3, CD4, CD8, and CD31 positive cells on days 0-21. Scale bars are S.D. of duplicate sections (n=4).

FIG. 23. NanoString analysis of subcutaneous K-luc tumors using the mouse immune panel. 750 mRNA concentration measurements, measured in baseline cells first at day 0 and then in subcutaneously transplanted tumors in separate mice at day 7, 14, and 21 (n=2). Sample 507 is a tumor that was not treated with ICG-001, was harvested on day 21, and is a baseline for comparison for days 7, 14, and 21. The log₁₀ fold-change in mRNA expression was considered between 507 and the other samples.

FIG. 24A is a table showing the top 20 upregulated genes in syngeneic subcut. Tumors. FIG. 24B is a table showing the genes that appear more than once. In FIG. 24A.

FIG. 25A is a table showing the top 20 downregulated genes in syngeneic subcut. Tumors. FIG. 25B is a table showing the genes that appear more than once. In FIG. 24A.

FIG. 26 are graphs showing changes in effects over the indicated timeframe according to some embodiments. Significant changes are shown in Day 7 and decreased throughout time. Note: y-axis is log2 scale, antigen proc, CD, dendr, INF, MHC.

FIG. 27. T cells treated with ICG001 2.5 and 5 μM for 24 or 72 h. (A) CAR Ts mock and HER2 from one donor and mock CARs derived from two additional donors were plated on 6-well format plates and treated with ICG001 at concentrations 0-5 μM for 24 or 72 h. Cells were collected from each well, counted, and viability was determined. (B, C) NanoString analysis of T cells treated with ICG-001 to determine concentration-dependent metabolic changes. (D) Heat map of up- and downregulated genes in T cells post ICG-001 treatment. (E) Correlation of differential gene expression in all T cell lines combined at concentration of 2.5 and 5 μM.

FIG. 28 is a table showing the top 20 most significantly up and downregulated genes at 2.5 μg and 5 μg dose in T cells derived from 3 donors.

FIG. 29 is a bar graph showing the results of studies where CAR T calls are treated with ICG001 for 24 hours according to some embodiments. To determine how treatment with ICG001 affects the T cells, the following method was employed. On day 1, CAR Ts HER 2 or Mock CARs derived from the same donor and additional 2 donor mock cells were plated on 6 well format plates and treated with ICG001 0 uM, 2.5 uM, 5 uM for 24-72 h. On day 5, cells were collected per well, counted, cell viability was determined [pellets were frozen]. The results indicate that ICG001 is not affecting Car T cell viability and proliferation. Note: CAR T from 494 donor treated with ICG001 were used in cytotoxicity assay #19 in comparison with untreated 4949 CARs.

FIG. 30 is a schematic showing the WNT Catenin Pathway, including the factors involved in the regulation of differentiation and proliferation via CBP/b-catenin arm of the WNT catenin pathway.

FIG. 31 is a schematic showing the selective inhibition of WNT pathway with CBP/catenin antagonist ICG-001.

FIG. 32 Proteomics analysis of PBT147 and PBT030 cell lines treated with ICG-001 (0, 5, 10 mM) for 72 h. Protein density was calculated to the difference in log normalized levels for each protein between the four treated samples and the appropriate untreated sample. Calculating the difference in log levels corresponds also to looking at the log of the fold changes in each protein. Differentially expressed proteins have been subjected to pathway enrichment analysis utilizing KEGG data base. Pathways with enrichment p-value <0.0001 in either 5 uM or 10 uM ICG001 treatments have been used for figures.

### DETAILED DESCRIPTION

The embodiments described herein include engineered chimeric antigen receptor (CAR)-modified T cells to stimulate anti-tumor immunity in combination with a small molecule Wnt/β-catenin pathway modulator. Activation of tumor-intrinsic Wnt/β-catenin signaling likely mediates cancer immune evasion by preventing T cell and dendritic cell infiltration, and thus resistance to immunotherapy. Thus, modulating Wnt/β-catenin signaling may enhance the response to CAR T cell therapy in the “cold” non-inflamed tumor microenvironment in relapsed and refractory patients. It is noted that while the methods described herein may be used for treating any tumor or cancer, some of the embodiments described below are directed primarily to treating brain tumors.

In certain embodiments, a method for treating a brain tumor is provided. In certain aspects, the method includes a step of administering a population of cells expressing a chimeric antigen receptor (e.g., a CAR-T cell that targets IL13Rα2 and/or HER2) in combination with a Wnt/β-catenin pathway modulator. The methods may be used to treat any type of brain tumor in an adult or pediatric setting including, but not limited to, glioma, ependymoma , medulloblastoma and other types of brain tumors (e.g., different histological types of brain tumors such as oligodendroglioma, astrocytoma, and the like).

CAR T cell immunotherapy is emerging as a promising strategy to treat many types of cancer and may offer new therapeutic options for patients diagnosed with refractory brain tumors. Adoptive transfer of (CAR) expressing T-cells, engineered to target tumor-specific antigens without need of antigen priming and independent of MHC restrictions, has demonstrated promising responses in clinical studies for brain tumors (Wang, Starr et al. 2020). Thus, the use of CAR-Ts to target tumors could facilitate selective targeting of brain tumors, providing a heretofore unachievable therapeutic specificity for malignant cells, while minimizing toxicity to surrounding tissue. However, antitumor function of CAR T therapy can be inhibited by adaptive resistance mechanisms (involved upregulation of IFNy expression), upregulation of immunosuppressive pathways and antigen escape (Brown and Mackall 2019). The limitations arising from heterogeneity of the tumor, low mutational burden, single antigen targeting and associated antigen escape are widely contributing to the tumor recurrence (Alizadeh, Wong et al. 2019, Bhaduri, Di Lullo et al. 2020). The tumor-associated antigens IL13Rα2 and/or HER2 are expressed by approximately 85-95% of PBT samples but are not expressed at significant levels in normal brain. Preclinical studies have optimized IL13Rα2- and HER2-CAR T cells for specificity, persistence, and antitumor potency, in adult patients with recurrent GBM. A pediatric patient with GBM was treated on an ongoing IL13Rα2-CAR T trial, and early clinical findings have established safety and indicated potential antitumor activity in adults, suggesting promise for this approach. But there are significant limitations to the CAR-T cells, as the bioactivity of the therapeutic CAR T cells is only observed in a subset of patients, and therapeutic resistance and relapse pose a significant problem.

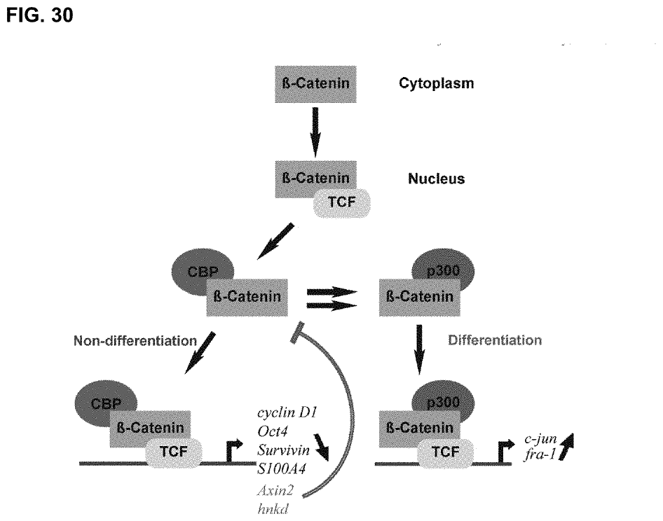

Clinical and preclinical data suggest that curative immunotherapy must not only address immunotolerance and target tumor antigens, but also circumvent evolving barriers of adaptive and acquired escape mechanisms. One mechanism of resistance involves activation of the Wnt/β-catenin signaling pathway (Luke, Bao et al. 2019), the Wnt/β-catenin pathway likely plays an important role in immune exclusion and as a possible resistance mechanism to CAR T therapy. The canonical WNT catenin pathway is shown in FIG. 30. Increased expression of β-catenin in tumors was inversely correlated with a lack of intratumoral CD8+ T cells and the presence of dendritic cells (Spranger, Bao et al. 2015). This hostile tumor microenvironment (TME) leads to loss of therapeutic efficacy to immunotherapy, tumor antigen vaccination, and adoptive T cell transfer immunotherapy (including CAR T cell) approaches (Gajewski, Corrales et al. 2017, Spranger, Dai et al. 2017, Horton, Williams et al. 2018, Osawa, Kojika et al. 2019). Clinical data with IL13Rα2-CAR T cells also support endogenous T cell tumor infiltration as a marker of response to CAR T cell therapy (Brown and Mackall 2019). Further support for the idea that Wnt/β-catenin activity has a role in response, was provided by a patient who had a complete response to CAR T therapy after initially presenting with a relatively high tumor mutation burden, T cell inflamed tumors, and low Wnt/β-catenin activity (Brown, Alizadeh et al. 2016). Progression of disease and relapse after CAR T therapy was associated with decreased mutational burden, tumor antigen escape, decreased immune infiltration, and Wnt/β-catenin activation in tumor cells.

According to some embodiments, the Wnt/β-catenin modulator used in the methods described herein is any suitable CPB/β-catenin antagonist. Suitable CPB/β-catenin antagonists include, but are not limited to, ICG-001 and PRI-724. It was previously demonstrated that small molecule CBP/β-catenin antagonist therapies can safely overcome radio-, chemo- and immunotherapy resistance by targeting a fundamental control switch in tumor stem/progenitor cells and inducing differentiation (Gajewski, Corrales et al. 2017, Spranger, Dai et al. 2017, Horton, Williams et al. 2018, Osawa, Kojika et al. 2019). Other studies have demonstrated that a change in the use of Kat3 coactivator from CBP/β-catenin- to p300/β-catenin-driven transcription, which can be accomplished pharmacologically by utilizing highly specific small molecule CBP/β-catenin antagonists (ICG-001; PRI-724 clinically evaluated analog), is associated with activation from a quiescent “stem-like” state to initiate differentiation of both normal somatic stem cells (SSC) and cancer stem cells (CSC). Specific CBP/β-catenin antagonists take advantage of the intrinsic preference of SSC to divide asymmetrically, whereas CSC preferentially divide symmetrically. Thus, CBP/β-catenin antagonist forced symmetric divisions of CSC can eliminate quiescent CSC without damaging the normal endogenous SSC population.

Wingless (Wg)-related integration site (Wnt) pathway activation in glioma has been associated with poor prognosis and progressive neurological deficits (Martin-Orozco, Sanchez-Fernandez et al. 2019) (Portella, 2020). Wnt signaling is associated with the proliferation of stem-like cells in human GBM (Kahlert, Bender et al. 2012, Kahlert, Maciaczyk et al. 2012, Kahlert, Suwala et al. 2015) and induces chemoresistance to chemotherapy and radio-therapy in glioblastoma (Paw, Carpenter et al. 2015, Huang, Zhang et al. 2020). β-catenin activation (translocation to the nuclei) is a downstream event of Wnt pathway activation. It has been identified in 19% of adult and in 30% of pediatric patient brain tumors (Holland 2000). Wnt pathway inhibition lead to suppression of tumor growth (Portela, Venkataramani et al. 2019). These data show that the network expansion and the accumulation of Fz1 in the TM projections may have an effect on neighboring neurons (Portela, Venkataramani et al. 2019). One possible mechanism of activation of Wnt in GBM tumor cells is via tumor microtubules (TM) which enwrap neurons and deplete Wg from neurons, while accumulating Wg receptor Frizzled1 (Fz1) on glioma cells. Activation of Wnt is causing tumor cell expansion, invasion by upregulation of JNK and accumulation of metalloproteases, while neurons degenerate because of Wnt extinction (Portela, Venkataramani et al. 2019). Tao et al. reported that GSC secrete the Wnt-induced signaling protein 1 (WISP1 ) to facilitate a pro-tumor microenvironment by promoting the survival of both GSCs and tumor-associated macrophages (TAMs). Silencing WISP1 markedly disrupts GSC maintenance, reduces tumor-supportive TAMs (M2), and potently inhibits GBM growth (Tao, Chu et al. 2020). WISP1 plays critical roles in maintaining GSCs and tumor-supportive TAMs in GBM, indicating that targeting Wnt/β- catenin-WISP1 signaling may effectively improve GBM treatment and the patient survival.

GSCs, comprising a small fraction of cancer cells at the apex of the differentiation hierarchy, play crucial roles in tumor initiation, cancer invasion, tumor angiogenesis, immune evasion, and therapeutic resistance (Kim, Dey et al. 2020) GSCs actively interact with other cells in the tumor microenvironment to promote malignant progression in GBMs. Thus, targeting GSCs and their interactions with other components of the tumor microenvironment has the potential for improving GBM treatment. Cancer stem cells (CSCs), also known as cancer-initiating cells, are associated with tumor initiation, growth, and metastasis. Growing evidence suggests that CSCs may be responsible for cancer therapy resistance and relapse or recurrence (Al-Hajj et al., 2003; Boumandi et al., 2014; Brooks et al., 2015; Prager et al., 2019; Saygin et al., 2019). To achieve complete regression of tumors, CSCs must be targeted based on the CSC theory (Chen and Wang, 2019) Growing evidence suggests that CSCs may secrete various growth factors and cytokines to inhibit immune responses and promote an immunosuppressive tumor microenvironment (Zhang et al., 2018; Prager et al., 2019; Clara et al., 2020). A previous report showed that tumor-intrinsic Wnt/β-catenin signaling could regulate the tumor microenvironment to promote malignant progression.

Activation of Wnt/β-catenin signaling in melanoma inhibits T cell infiltration to promote tumor growth and therapeutic resistance by regulating CCL4 secretion. In GBMs, Wnt/β-catenin signaling is highly activated in GSCs, promoting malignant transformation and tumor progression. Wnt/FZD signaling is frequently up-regulated in GBM (Kahlert, Suwala et al. 2015, Suwala, Hanaford et al. 2016, Suwala, Koch et al. 2018). In particular, one of the hallmarks of bad prognosis is the accumulation of β-catenin in tumor cells, indicating an activation of Wnt/FZD pathway (Portela, 2020).

Further, a patient with GBM who initially had a complete response to CAR T therapy after presenting with relatively high tumor mutational burden, T cell inflamed tumors, and low Wnt/β-catenin activity (Gajewski, Corrales et al. 2017). However, after CAR T therapy, the patient's disease progressed and relapsed, which was associated with decreased mutational burden, tumor antigen escape, decreased immune infiltration, and enhanced Wnt/β-catenin activation in tumors (Brown, Alizadeh et al. 2016).

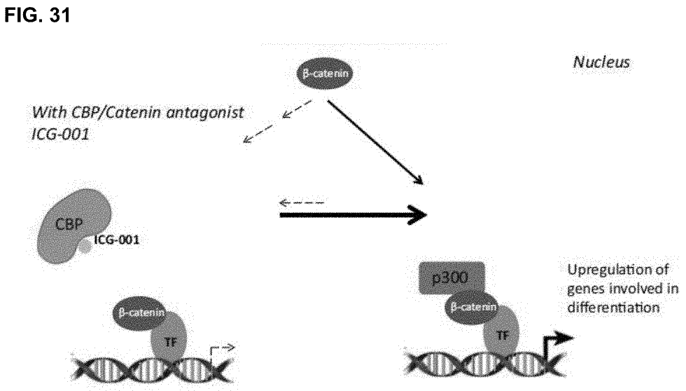

Additionally, it has been shown that small molecule CREB binding protein CBP/β-catenin antagonist therapies can safely overcome radio-, chemo- and immunotherapy resistance by targeting a fundamental control switch in tumor stem/progenitor cells that induces differentiation (Gajewski, Corrales et al. 2017, Spranger, Dai et al. 2017, Horton, Williams et al. 2018, Osawa, Kojika et al. 2019). A change in the use of Kat3 coactivator from CBP/β-catenin- to p300/β-catenin-driven transcription, which can be accomplished pharmacologically by using highly specific small molecule CBP/β-catenin antagonists (i.e., ICG-001; PRI-724 clinically evaluated analog of ICG-001 ) was associated with activation from a quiescent “stem-like” state to initiate differentiation of both normal somatic stem cells (SSCs) and cancer stem cells (CSCs) (Thomas and Kahn 2016, Manegold, Lai et al. 2018, Lai, Nguyen et al. 2019). ICG-001 selectively blocks the β-catenin/CBP interaction without interfering with the β-catenin/p300 interaction (FIG. 31).

Specific CBP/β-catenin antagonists take advantage of the intrinsic preference of GSCs to divide asymmetrically, whereas CSCs preferentially divide symmetrically (Lukaszewicz, Nguyen et al. 2019). Thus, CBP/β-catenin antagonist-forced symmetric divisions of CSCs can eliminate quiescent CSCs without damaging the normal endogenous SSC population(Zhao, Masiello et al. 2016). In preclinical studies, ICG-001 has ameliorated therapy-induced toxicity through beneficial effects on the normal somatic stem cell population (Spranger, Bao et al. 2015, Gajewski, Corrales et al. 2017, Spranger, Dai et al. 2017). This may be beneficial for preventing therapy-induced developmental defects in young children with BTs.

The combination approach described herein can be rapidly translated to the clinic to benefit children with PBTs (as well as adults), as both CAR T therapy and CBP/β-catenin antagonists, as single agents and in combination with chemotherapy, have been proven safe and efficacious in clinical trials. Furthermore, the information gained on utility and effects on the TME of combination therapy with ICG-001 and CAR T cell therapy may also be used to identify which patients would most benefit from this therapy, leading to improved personalized treatment for BTs that can be translated to other cancers.

In summary, previous studies have demonstrated the safety and antitumor activity of IL13Rα2-specific CAR T cells for the treatment of recurrent adult glioma (Wang et al. 2014; Joshi et al. 2008). However, clinical and preclinical data suggest that curative immunotherapy must not only address immunotolerance and target tumor antigens, but also circumvent evolving barriers of adaptive and acquired escape mechanisms. A common mechanism of resistance observed in immunologically “cold tumors” (such as PBTs) involves activation of the Wnt/β-catenin signaling pathway (Luke et al. 2019; Gajewski et al. 2017)). Tumor-intrinsic enhanced Wnt/β-catenin signaling appears to be a common mechanism mediating cancer immune evasion, associated with the presence of an immunosuppressive cell subset and prevention of effective dendritic cell presentation and T-effector cell recruitment and function. Increased expression of β-catenin is inversely correlated with the presence CD8+ T cells and dendritic cells in brain tumors (Gajewski et al. 2017; Spranger et al. 2015). This hostile tumor microenvironment (TME) leads to loss of efficacy of immunotherapy, tumor antigen loss, and adoptive T cell transfer immunotherapy (including CAR T cell) approaches (Brown et al. 2015; Brown et al. 2016; Priceman et al. 2018; Brown et al. 2018). Other clinical data with IL13Rα2-CAR T cells support endogenous T cell tumor infiltration as a marker of response to CAR T cell therapy in GBM (Brown et al. 2016). Therefore, downregulation of Wnt/β-catenin signaling should enhance the response to CAR T therapy in the “cold” non-inflamed TME (lacking infiltration of T cells) in patients with relapsed and refractory brain tumors. Further, small molecule CBP/β-catenin antagonist therapies can safely overcome radio-, chemo- and immunotherapy resistance by targeting a fundamental control switch in tumor stem/progenitor cells that induces differentiation (Gajewski et al. 2017; Spranger et al. 2017; Horton et al. 2018; Osawa et al. 2019).

In the studies described below, it was determined the extent to which the highly specific, small molecule Wnt/CBP/β-catenin antagonist ICG-001 can modify glioma tumor cells and immune infiltration, thus contributing to overcoming resistance to therapy in BTs. The cytostatic effect of ICG-001 on multiple glioma cell lines, derived from patient derived (PDX) and murine tumors (GL261, K-Luc), was demonstrated. Downregulation of target genes Survivin/BIRC5 (CBP/catenin dependent) and upregulation of EphB2 (p300 dependent) was observed, demonstrating the selective inhibition of CBP/catenin signaling in these lines. The effect of ICG-001 in vitro on selected patient-derived cell lines was also characterized using Nanostring and Proteomics analysis, elucidating pathways involved in WNT signaling. Furthermore, ICG-001 treatment in syngeneic mouse model of glioma (K-luc) has been demonstrated to induce CD8 cells infiltration and increased expression of CD31. Therefore, addition of ICG-001 may potentiate glioma cell differentiation in vivo and in vitro and can induce immunotherapy interventions in glioma patients.

Moreover, while some studies show encouraging results in a subset of cancers using checkpoint immunotherapies including anti-CTLA4, anti-PD-1 and anti-PD-L1 or CAR-T therapies (Sharma et al. 2021), the heterogeneity of tumors, low mutational burden, single antigen targeting, and associated antigen escape widely contribute to tumor recurrence Miranda et al. 2019, Malta et al, 2018). An important common mechanism of resistance that is observed in immunologically “cold tumors”, including gliomas, involves aberrant activation of Wnt/β-catenin signaling (Luke et al. 2019, Gajewski et al. 2017). Enhanced tumor-intrinsic Wnt/β-catenin signaling appears to be a common mechanism mediating cancer immune evasion and is associated with the presence of an immunosuppressive cell subset and prevention of effective dendritic cell presentation and T-effector cell recruitment and function (Tao et al. 2020). Increased expression of β-catenin inversely correlates with the presence of CD8+ T cells and dendritic cells in multiple tumor types including glioma (Gajewski et al. 2017, Spranger et al. 2015). Furthermore, Wnt pathway activation is correlated with tumor stemness and poor outcome (Chen et al., 2021, Wiese et al. 2020). The hostile tumor microenvironment (TME) is associated with decreased tumor antigen presentation and greatly reduced or lost efficacy of various therapies, including adoptive T cell immunotherapy (Brown et al. 2015, Brown et al. 2016, Priceman et al. 2018, Brown et al. 2018, Zhang et al. 2013). Therefore, targeting downregulation of Wnt/β-catenin signaling and thereby enhancing the response to immunotherapy in patients with relapsed and refractory tumors is an attractive therapeutic approach. In the studies below, the effects of the highly specific, small molecule Wnt/CBP/β-catenin antagonist ICG-001 on glioma tumor cells and the TME was determined, including immune cell infiltration, blood vessel decompression and metabolic changes. Differentiation of glioma cells, with a loss of glioma stem cells, can enhance the antitumor immune response. In vitro cytostatic effects and a switch from proliferation to differentiation after treatment was demonstrated with ICG-001, using multiple patient-derived (PDX) glioma cell lines and murine tumors (GL261, K-Luc). It was further demonstrated that, in these glioma cell lines, ICG-001 down-regulated the CBP/β-catenin target gene survivin/BIRC5, a hallmark of selective inhibition of CBP/β-catenin signaling. Utilizing Nanostring and proteomics analysis, the effects of ICG-001 in vitro on the patient derived glioma cell lines PBT030 and PBT147 were investigated, as well as metabolic pathways regulated by WNT signaling. Furthermore, in a syngeneic mouse model of glioma (K-luc), ICG-001 treatment enhances tumor infiltration by CD3+, CD4+ and CD8+ cells and increased expression of vascular marker CD31. Consequently, reprogramming the glioma tumor microenvironment, via differentiation of both the tumor, stroma and immune cell recruitment utilizing the specific Wnt/CBP/β-catenin antagonist ICG-001 may enhance immunotherapy interventions in glioma patients.

The following examples are intended to illustrate various embodiments of the invention. As such, the specific embodiments discussed are not to be construed as limitations on the scope of the invention. It will be apparent to one skilled in the art that various equivalents, changes, and modifications may be made without departing from the scope of invention, and it is understood that such equivalent embodiments are to be included herein. Accordingly, the invention is not limited except as by the appended claims. Further, all references cited in the disclosure are hereby incorporated by reference in their entirety, as if fully set forth herein.

### EXAMPLES

Because activation of tumor-intrinsic Wnt/β-catenin signaling appears to be a common mechanism mediating cancer immune evasion by preventing T cell and dendritic cell infiltration, and thus resistance to immunotherapy, downregulation of Wnt/β-catenin signaling with highly specific small molecule should enhance the response to CAR T cell therapy via modulation of blood flow (inhibition of renin angiotensin system) and extracellular matrix remodeling (antifibrotic obstructive effect), elimination of CSCs and improved immune cells infiltration. Preliminary studies indicate that treatment of patient-derived tumors with Wnt selective inhibitor induces differentiation in multiple glioma cell lines in vitro, upregulation of target genes and synergy in killing glioma when combined with CAR T cells. In summary, the studies described below demonstrate the clinical feasibility of elimination of extrinsic and intrinsic determinants of immunotherapy resistance in glioma by combining Wnt modulating agent with approved CAR T cell therapy. Importantly, upon establishing pre-clinical proof-of-concept, this combinational approach could be rapidly translated to the clinic.

These studies build on clinical experience demonstrating safety and transient antitumor activity with the IL13Rα2- and HER2-specific CAR T cell platforms, and now incorporate several innovations in CAR design and T cell manufacturing that have resulted in improved persistence and therapeutic efficacy in preclinical models of adult glioma and in an ongoing IL13Rα2-CAR T clinical trial. A second generation IL13Rα2-specific CAR T cell platform, termed IL13BBzeta, was developed and that incorporates the 4-1BB (CD137) co-stimulatory domain and a manufacturing platform using enriched central memory T cells, which improves memory T cell survival and maintenance. Using human glioma xenograft models with patient-derived tumors in NSG mice, it was found that IL13BBzeta-CAR T cells improved anti-tumor activity and T cell persistence as compared to first-generation IL13zeta-CAR CD8(+) T cells that had shown evidence for bioactivity in patients(Brown, Aguilar et al. 2018). To overcome known limitations of use of CAR T therapy for brain tumors, such as antigen escape and need for increased T cell persistence and potency, a novel combinational therapy approach with the Wnt/β-catenin antagonist ICG-001 may be used.

Establishing proof-of-concept that ICG-001 can safely target aberrant Wnt/β-catenin signaling in PBTs in combination with CAR T cell therapy could provide a novel way of broadening tumor response to CAR T therapy while decreasing resistance and relapse arising post-CAR T therapy in solid tumors. The immune subsets and Wnt/β-catenin pathway activation in the tumor microenvironment will also separately analyzed in PBT tumors in preclinical models using novel techniques such as NanoString and Seahorse analysis and will correlate these results with those from patient samples obtained post-CAR T therapy, including outcome. Tumor heterogeneity in terms of antigen expression also remains a major challenge to use of CAR T cell therapy for solid tumors, and new approaches are needed to overcome this complication. CBP/β-catenin antagonists, by enhancing the immunostimulatory tumor microenvironment, should enhance antigen cross presentation by professional antigen presenting cells (e.g., BATF3⁺ dendritic cells) to generate endogenous B- and T-cell immunity against tumor antigens not targeted by the CAR T cells themselves (Staal, Luis et al. 2008, Spranger, Dai et al. 2017). ICG-001 should also modify the “cold” TME and change the mode of division of CAR T cells, increasing the T-memory cell population thereby further preventing resistance and relapse.

### Example 1: Wnt/β-Catenin Pathway Activation is Associated with Tumor Relapse and Antigen Loss

Immune exclusion via Wnt/β-catenin signaling plays an important mechanistic role in CAR T therapy resistance, and increased expression of β-catenin in tumors is inversely correlated with a lack of intratumoral CD8⁺ T cells and dendritic cells. Therefore, CAR T therapy should be even more powerful when combined with a Wnt/CBP/β-catenin antagonist (e.g., ICG-001 or PRI-724 [the clinically evaluated analog of ICG-001]). The extent to which the highly specific small molecule CBP/β-catenin antagonist ICG-001, in combination with CAR T immunotherapy, can overcome therapy relapse to effectively target refractory/resistant PBTs will be tested. An established patient-derived xenograft (PDX) models and a murine CAR and syngeneic mouse platform will be used to evaluate the synergy of CAR T cell therapy with ICG-001 and the impact of CBP/β-catenin inhibition on the TME (CD8⁺, CD4⁺, T_{eff} vs. T_{reg}, MDSCs, M1 vs. M2 macrophages) (Youngblood, Hale et al. 2017). Flow cytometry and immunohistochemistry (IHC) analyses will be used to determine the effect of the combination of ICG-001 with CAR T cells on improving T cell infiltration (CD8⁺), dendritic cell recruitment, T_{reg} down-regulation, and antitumor activity. The number of CD133⁺ CSCs in each treatment group will be determined to assess toxicities, monitor tumor burden via bioluminescent imaging, and track long-term survival in preclinical models.

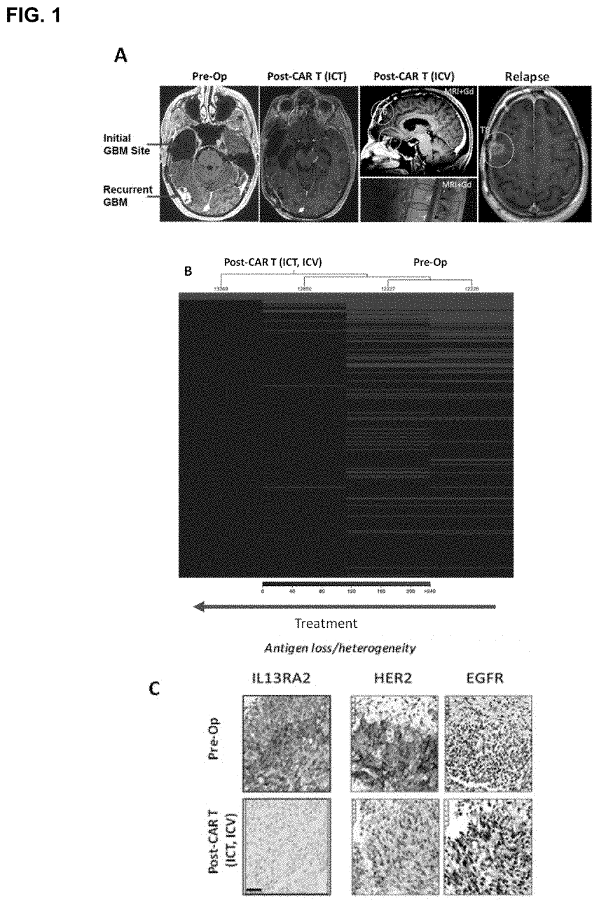

These studies will be performed in PDX models of pediatric EP and glioma, which mimic the clinical progression of these cancers in terms of ventricular spread and dissemination via cerebrospinal fluid (CSF) flow. Preliminary studies using data from an ongoing IL13Rα2-CAR T clinical trial indicated that Wnt/β-catenin pathway activation is associated with tumor relapse and antigen loss, as detected by RNA-seq and IHC (FIG. 1) (Brown, Alizadeh et al. 2016).

Given the Wnt/β-catenin pathway activation seen after CAR T cell treatment, it was determined if the CBP/β-catenin antagonist ICG-001 could enhance CAR T cell-mediated tumor cell killing. To do this, in vitro cytotoxicity assays were used as discussed below.

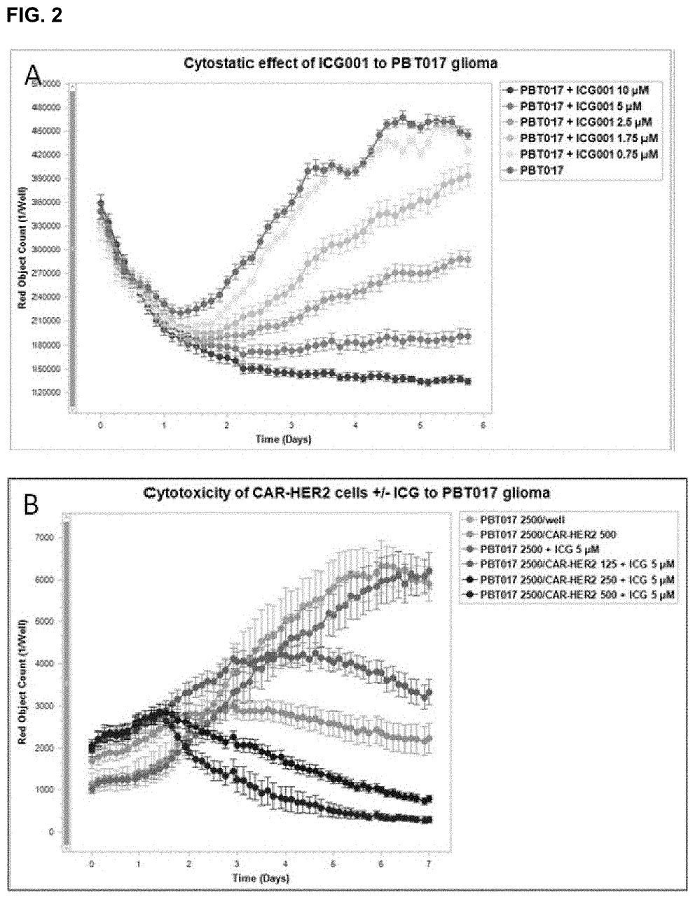

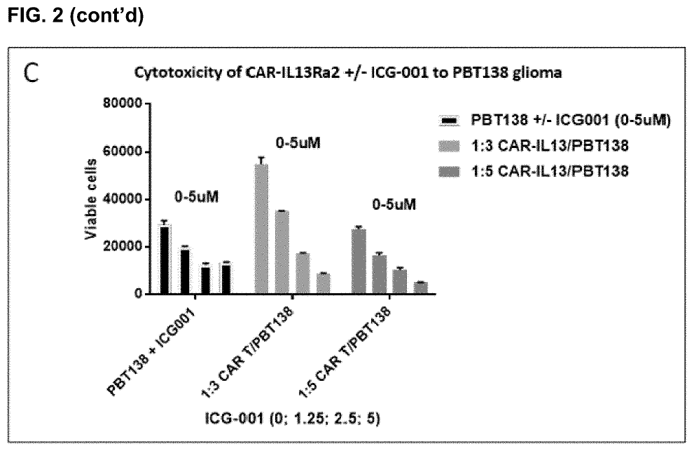

Cytotoxicity assays testing ICG-001 only (FIG. 2A) or in combination with HER2-CAR T Cells (FIG. 2B) or IL13Rα2-CAR T cells (FIG. 2C) demonstrated synergistic killing of PBT017 and PBT138 patient tumor-derived glioma cells (lines that express high levels of the tumor antigens IL13Rα2 and HER2). It has also been observed that ICG-001-induced differentiation of PBT017 and PBT138 (data not shown). For the studies described herein, a unique repository of previously established PDX lines (ependymoma, medulloblastoma, and glioma) will be used, using representative lines exhibiting low and high expression of IL13Rα2 and HER2 for each type of tumor for in vitro and in vivo experiments in Example 2 below.

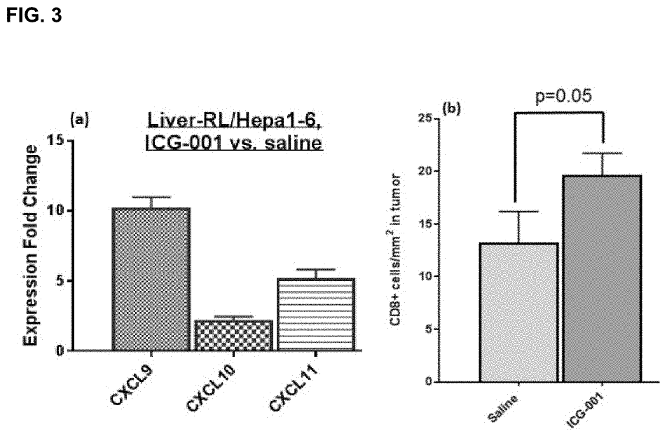

It was previously demonstrated, in an orthotopic syngeneic murine hepatocellular carcinoma model, that ICG-001 increased the expression of the interferon-y regulated chemokines CXCL9-11 in the TME (FIG. 3A). CXCL9 and CXCL10, are produced mainly by BATF3 dendritic cells that are required for T_{eff} trafficking. The CXCL9, CXCL10, CXCL11/CXCR3 signaling axis is critical for activated T-helper type 1 (Th1), cytotoxic T-cell (CTL), and natural killer (NK) cell recruitment immune cell recruitment in vivo. A similar effect resulting from ICG-001 is likely, such as increased CD8⁺T-cell infiltration into brain tumors (FIG. 3B).

It has also been shown that treatment with ICG-001, via a concomitant increase in p300/β-catenin transcription, is associated with tumor cell differentiation and a decrease in glycolytic activity and extracellular acidification due to changes in tumor cell metabolism. This creates a TME that is more conducive to T_{eff} cells and less so to T_{reg} cells and other immune cells with a suppressive phenotype (e.g., MDSCs) (Thomas and Kahn 2016, Spranger, Dai et al. 2017).

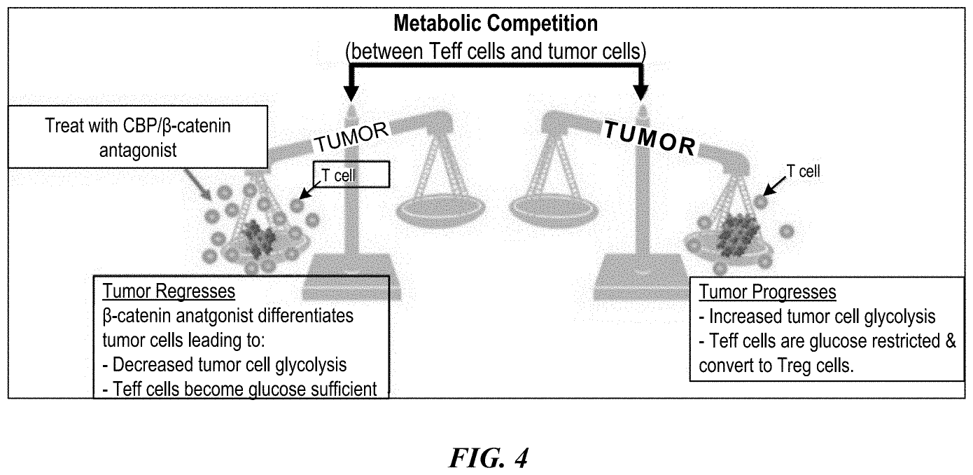

Modulation of target antigen density has been demonstrated to affect CAR T cell functionality and persistence, (Ramakrishna, Highfill et al. 2019) and ICG-001-increased antigen expression in glioma cells should further improve CAR T response (FIG. 4). Tumor heterogeneity and lack of target antigen expression are two major hurdles that need to be effectively dealt with in order for solid tumor CAR T therapy to achieve similar success to CAR T therapies for hematologic malignancies.

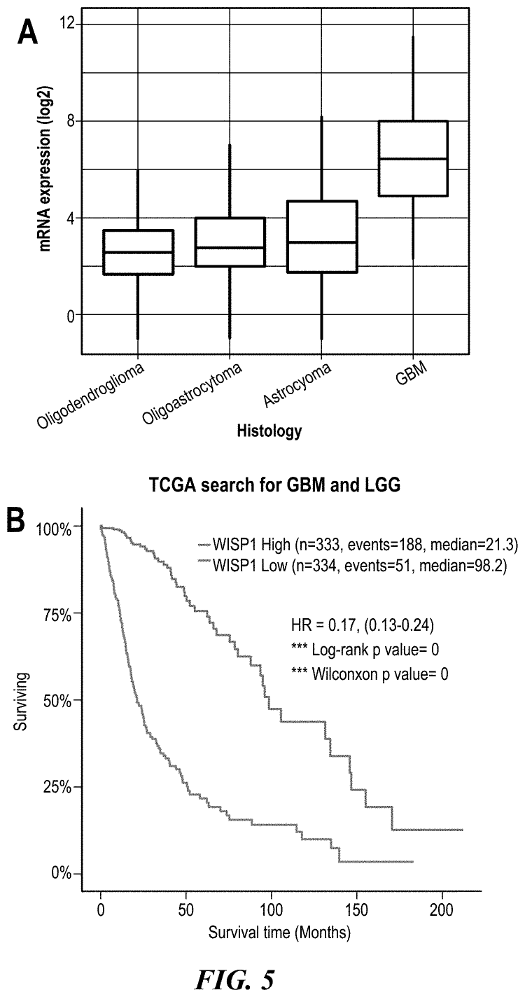

The Wnt/β-catenin pathway and its target genes such as WISP1 are upregulated in glioma stem cells (GSCs) and are linked to tumor microenvironment (Tao 2020). It was demonstrated, using GlioVis TCGA database search, that WISP 1 was highly expressed by low grade (LGG) and high grade gliomas (GBM), as compared with normal brain tissue and that WISP1 gene (biomarker for the Wnt pathway) expression correlated with poor patient survival (FIG. 5A, B).

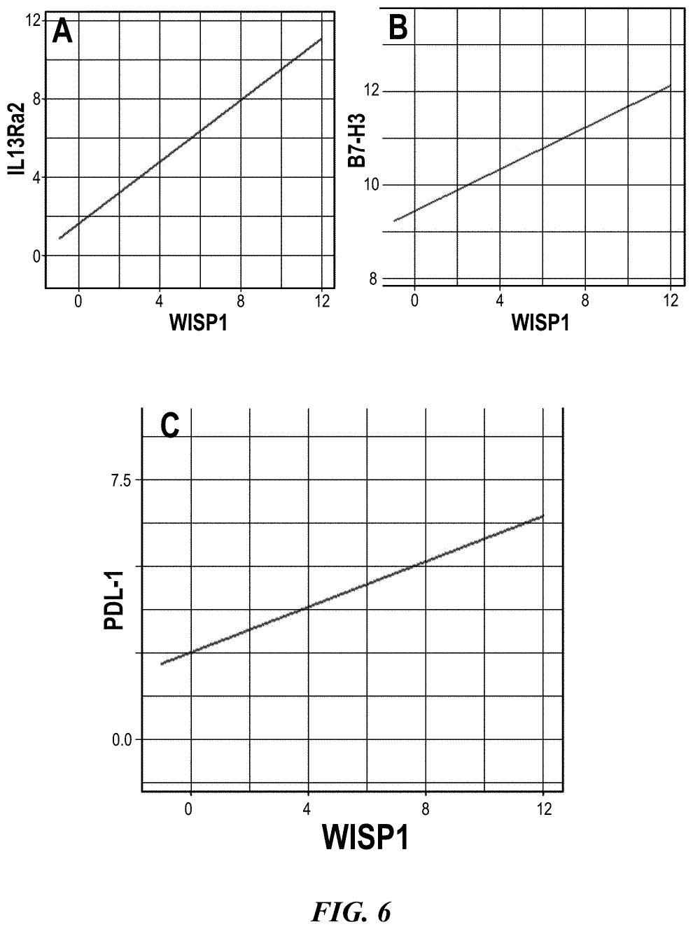

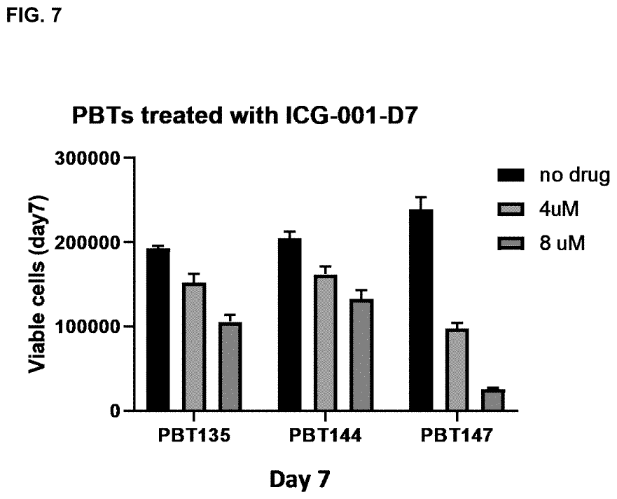

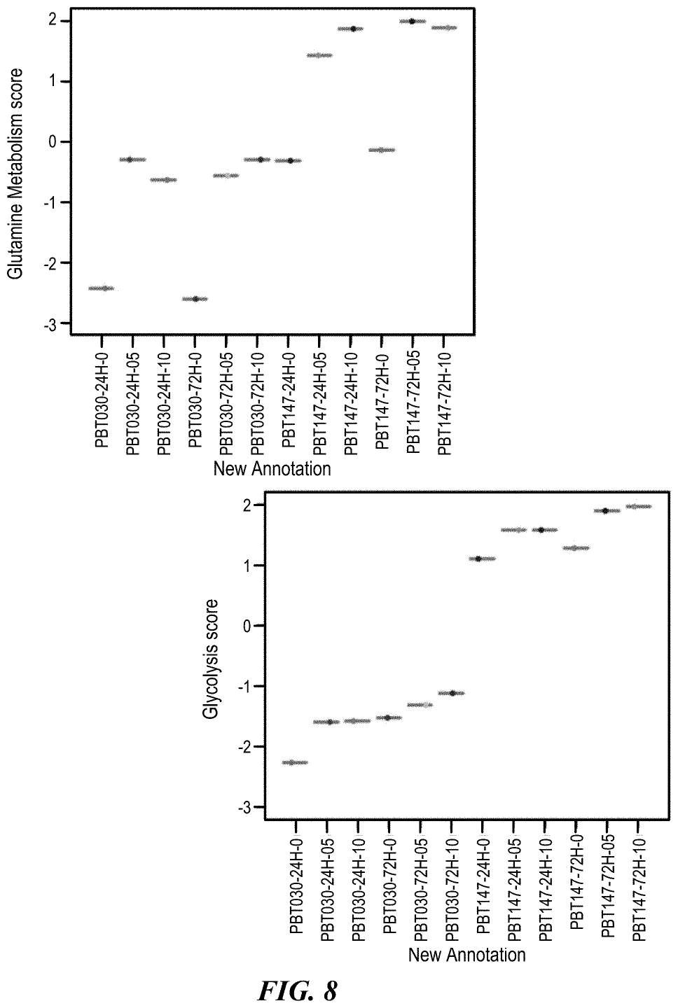

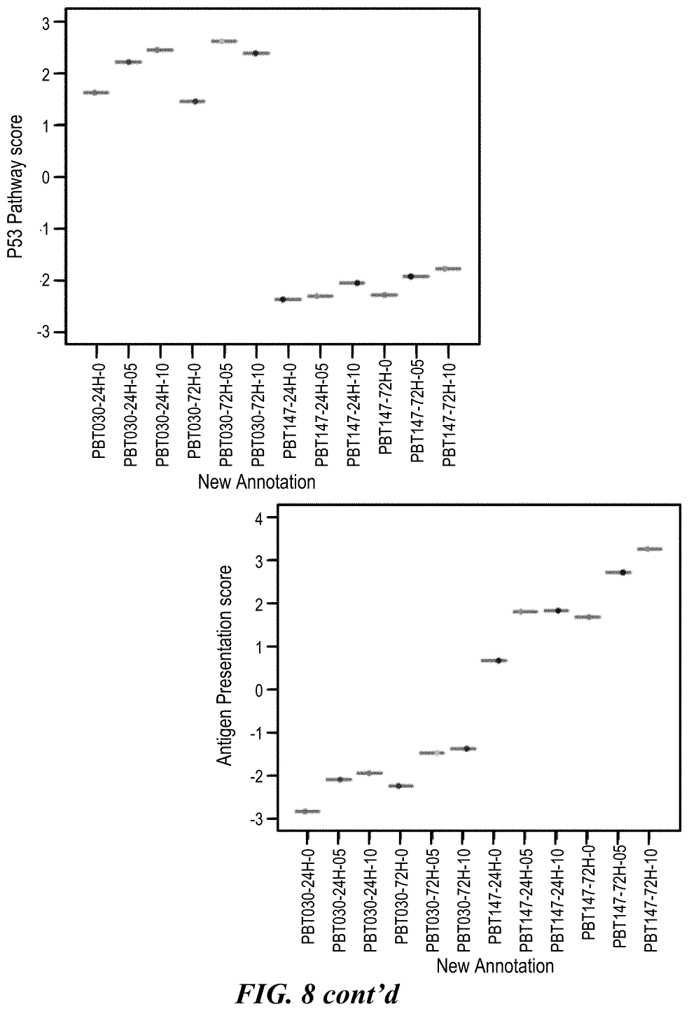

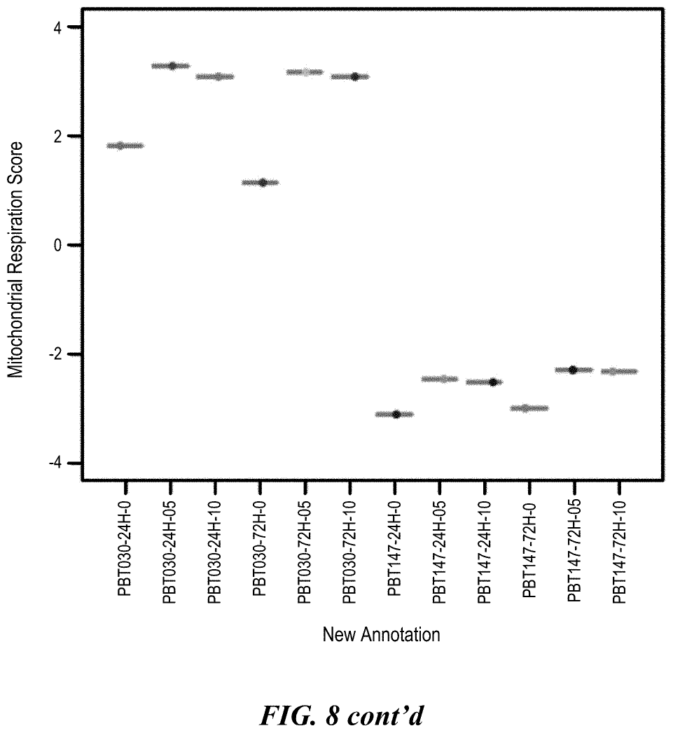

In addition, activation of Wnt/β-catenin and subsequent upregulation of WISP1 was strongly correlated with expression of tumor-specific antigens such as IL13Rα2, B7-H3, and PDL-1, as well as with EGFR, BIRC5-survivin, CTNNB1 and CD47 (FIG. 6 and data not shown).

PBT147 and PBT030 PBT cells were treated with ICG-001 (0, 5, or 10 μM) for 24-72 h. RNA expression was then analyzed using the NanoString nCounter platform (NanoString Technologies) by digitally detecting and counting in a single reaction without amplification using the NanoString human metabolic panel (City of Hope Molecular Pathology Core) (Geiss, Bumgarner et al. 2008). The data indicates change in a several metabolic markers and antigen presentation increased by PBT cells lines after treatment with ICG-001 at both time points. In addition, a cell cycle decrease indicating a possible differentiation switch was observed. Fatty acid oxidation which is a preferred metabolic pathway for “quiescent stem like cells” was decreased in all treated cells and all timepoints. An increased p53 pathway and mitochondrial respiration was also observed at both time points and both cell lines.

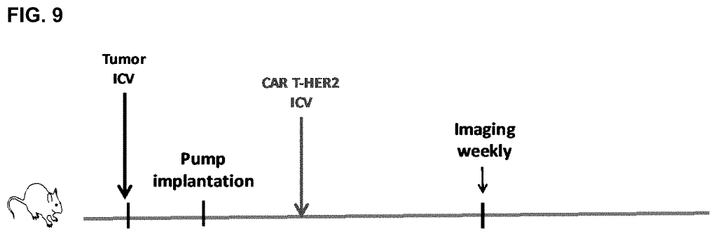

In vivo studies using PBT 138 cell line orthotopic models were demonstrated by IHC with KI-67, human Nestin and Cd3 staining (FIG. 9)

Examples 2 and 3 below will determine the extent to which the highly specific, small molecule Wnt/CBP/β-catenin antagonist ICG-001, in combination with CAR T immunotherapy, can overcome therapy relapse to effectively target refractory/resistant brain tumors. ICG-001 may overcome PBT tumor resistance and synergize with CAR T cell treatment via three mechanisms: 1) by eliminating [by differentiating CSC] cancer stem cells (CSCs), 2) by modifying the “cold” TME, and 3) by changing the mode of division of CAR T cells and increasing the T-memory cell population.

### Example 2: Preclinical Therapeutic Efficacy of CAR T Therapy in Combination with Small Molecule CBP/β-Catenin Antagonist ICG-001 in NSG Models In Vitro and In Vivo (Prophetic)

This study will determine the preclinical therapeutic efficacy of CAR T therapy in combination with the small molecule CBP/β-catenin antagonist ICG-001 in vitro and in vivo. In vitro cytotoxicity assays will be used to test if CSCs are more efficiently eliminated by CAR T cells when used in combination with ICG-001. These studies will use previously established patient-derived tumor sphere lines with CSC-like tumor-initiating features 15,16 and measure tumor killing with titrated doses of CAR T cells±ICG-001. In addition, human patient-derived xenograft [PDX] brain tumor models with high and low expression of either a single antigen (IL13Rα2 or HER2), or their combination, will be used to evaluate the in vivo efficacy of CAR T therapy±ICG-001 in NSG mice. HER2, IL13Rα2, EGFR, PD-L1 and CD133 expression will be characterized by the PBTs post therapy using flow cytometry and immunohistochemistry (IHC) analyses.

For in vitro studies, the efficacy of a titrated dose of CAR T cells±ICG-001 will be determined in in vitro cytotoxicity assays. These studies will use the following PDX GBM lines: PBT017 (HER2-positive) and two versions of the PBT138 cell line engineered to express low and high levels of IL13Rα2. Tumor cells will be plated in a 96-well format for 24 h and treated with CAR T cells (for IL13Rα2- or HER2-targeting as appropriate; 1:5; 1:10; 1:20) and ICG-001 (at 0-10 μM) in quadruplicate. Cell viability will then be assessed over 7 days using IncuCyte live cell imaging. These studies will define the minimum effective dose for IL13Rα2- or HER2-CAR T cells±ICG-001 needed to achieve synergistic killing of PBT cells, expressing low or high levels of tumor-specific antigen. Residual tumor cells will be analyzed by flow cytometry for the presence of CSC markers (CD133, CD44, MDR1, and GPD1) and CAR T-specific antigens (IL13Rα2 and HER2) (Rusu, Shao et al. 2019).

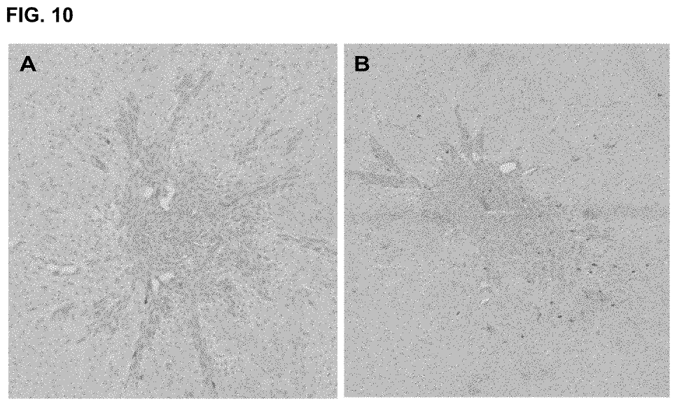

For in vivo studies, tumor growth and long term survival of mice bearing PDXs of glioma or ependymoma and treated with CAR T cells±ICG-001 will be measured. NOD-scid IL2Rγ^{null} (NSG) mice will be stereotactically implanted either intracranially (IC, for glioma) or intraventricularly (ICV, for EP) using established methods (Taylor, Poppleton et al. 2005). Established tumors (Day 10 post-implantation) will be treated ICV with a single infusion of IL13Rα2-CAR T cells or HER2-CAR T cells at titrated doses of 0.1, 0.25, and 0.5×10⁶±ICG-001 (50 mg/kg, administered via continuous infusion using subcutaneous Alzet minipumps). ICG-001 has been previously shown to cross the blood-brain barrier(Tran and Zheng 2017). Tumor regression and growth will be assessed by weekly Xenogen imaging of tumors and survival by Kaplan-Meier analysis. T cell tumor infiltration and persistence will be quantified by anti-CD3 (CD4 and CD8) IHC, and localization of CAR T cells within the tumor environment will be examined using IHC staining for T cells at day 10-14 post-CART cell administration, and at death or day 60 for surviving mice (n=5 mice per group). An example of the CD8+ staining of PDX human glioma and syngeneic murine glioma is shown in FIG. 10. Treatment groups for each model will be: 1) tumor only; 2) ICG-001 treatment only; 3) CAR T cell treatment only; 4) ICG-001+CAR T treatment (sub therapeutic dose of CAR Ts will be titrated and used for each tumor). ICG-001 will be given 2 days prior to CAR T cell administration to provide time for TME changes that may modulate activity of CAR T cell-mediated therapy.

Anti-tumor activity of CAR T therapy against high-expressing IL13Rα2/HER2 tumors is to be measured. ICG-001-treated tumors may demonstrate increased expression of IL13Rα2/HER2 and decreased expression of CSC markers by flow cytometry, thereby enhancing CAR T cell mediated therapy. Alternatively, ICG-001 can be given following CAR T administration, if ICG-001 given prior to CAR T therapy is not effective.

Statistical analyses will be performed separately for each treatment regimen depending on the route of administration and model (EP or glioma). All statistical analyses will be done using SAS version 9.4 and Prism version 6. Tumor growth will be monitored by Xenogen bioluminescent imaging, and mean signal intensities will be obtained from scans at multiple time points for each mouse. Results will be analyzed using random intercept and slope regression models with intensities analyzed on a log scale. Models will include quadratic time, group, and their interactions. Significant estimated group differences from curves will be analyzed using t-tests. In addition, t-tests on raw data will be used to confirm earliest time point of difference. Kaplan-Meier curves will be created in SAS version 9.4 and median survival with 95% confidence intervals will be estimated. Survival curves obtained in Aim 1 will be compared using log-rank test. Pairwise comparisons will be made with Sidak for multiple comparisons.

### Example 3: Effect of CBP/6-Catenin Inhibition on the Tumor Immune Microenvironment and Wnt/β-Catenin Pathway Activation in the Context of CAR T Therapy in a Syngeneic Glioma Model (Prophetic)

This study will determine the effect of CBP/β-catenin inhibition on the tumor immune microenvironment and Wnt/β-catenin pathway activation in the context of CAR T therapy in a syngeneic glioma model. An established murine CAR and syngeneic glioma platform will be used to determine the effect of CBP/β-catenin inhibition with ICG-001 on the GBM tumor microenvironment (CD8+, CD4+, Teff vs. Treg, myeloid-derived suppressor cells, M1 vs. M2 macrophages) and on efficacy of CAR T cell therapy. Preclinical findings related to gene expression data in the tumor TME with The Cancer Genome Atlas (TCGA) database for human BTs will be correlated with existing patient survival/histology/single cell RNA seq data. Flow cytometry, IHC, NanoString analysis, 10× transcriptomics, and metabolomics analysis will be used to determine whether the combination of ICG-001 with CAR T cells improves T cell infiltration (CD8+), dendritic cell recruitment, Treg down-regulation and antitumor activity.

Mouse models. Previously described immunocompetent mouse orthotopic glioma tumor models (K-Luc and GL261, engineered to express II13Rα2) will be used to determine the therapeutic potential of CAR T cells±ICG-001 therapy. Mice bearing stablished tumors (day 7-10 post-implantation) will be treated either intratumorally or ICV with a single infusion of IL13Rα2- or HER2-CAR T cells at titrated doses (0.5 and 1.0×10⁶ CAR T cells) ±ICG-001 (50-100 mg/kg), to define the minimum effective dose for each route using previously established methods (Kahlon, Brown et al. 2004, Wang, Berger et al. 2011). The advantage of these models is that they will allow us to investigate the effects of CBP/β-catenin antagonism on the tumor cells, the TME, and immune cell influx into the TME to elucidate the role of endogenous immune response. Experiments will include 8-14-week-old male and female mice to address potential sex differences (n=10 mice per group). Control groups will receive no treatment, or ICG-001 or CAR T treatment alone (sub-therapeutic dose). Treatment groups will receive ICG-001+ CAR T at sub-therapeutic doses. Tumor regression and growth will be assessed by weekly Xenogen imaging; IHC assessment of tumor infiltration by CD3, CD4, and CD8 cells; and survival by Kaplan-Meier analysis. The extent of T cell tumor infiltration and persistence in the brain also will be quantified by flow cytometry at days 1 and 10 post treatment, and at death or day 60 for surviving mice.

Evaluation of the TME. Tumor immune cell infiltration will be assessed using flow cytometric analysis of fresh tissues and validated using IHC. Changes in T cells (CD3+, CD8+, T_{regs}), tumor-associated macrophages, and MDSCs will be evaluated. Tumors also will be evaluated biochemically for the effect of CART cell therapy ±ICG-001, using a co-immunoprecipitation assay to evaluate CBP/β-catenin versus p300/β-catenin association, as previously described (Emami, Nguyen et al. 2004). GFP+tumor cells will be sorted by FACS and analyzed by RT-qPCR for changes in gene expression of survivin/BIRC5, Let7a, and by FACS for CD133 expression, as these genes are involved in establishing the CSC phenotype and are direct targets of ICG-001. Changes in gene expression in the TME will be assessed by RT-PCR for TGF-β, PAI-, GPD1 as well as the chemokines CXCL9, CXCL10, and CXCL11, which are part of a signaling axis critical for activated Th1, CTL, and NK cell recruitment.

Tumor immune cell infiltration and differentiation markers will be analyzed using single-cell immunochemistry (flow cytometry, light microscopy) and single-cell RNA expression profiling (using a human CAR T panel, NanoString)(Geiss, Bumgarner et al. 2008). The NanoString CAR T characterization panel consists of 770 genes that are known to predict CAR-T activity and 10 internal reference genes for data normalization. RNA will be hybridized with the Codeset from the gene panel at 65° C. for 16 h. The post-hybridization probe-target mixture will be quantified using the nCounter Digital Analyzer, and all data analysis will be performed on nSolver (NanoString Technologies). All raw data will be first normalized with internal positive and negative controls to eliminate variability unrelated to the samples, then normalized to the selected housekeeping genes using Geometric Means methods. The metabolic effects of CAR T+ICG-001 treatment will also be tested via Seahorse analysis during the course of differentiation and a decrease in glycolytic activity and extracellular acidification using an XFe96 metabolic analyzer (Neville, Bosse et al. 2018). Tumors from each treatment group will be isolated and analyzed: 1) tumor only; 2) ICG-001 treatment only; 3) CAR T only; 4) CAR T+ICG-001 will be selected (n=10) at 10-14 days or day 60 post CAR T treatment and will be compared with controls.

NanoString analysis will be performed as described above.

10× transcriptomics and single cell RNA seq: The relationship between cells and their organization within tissue is critical to understanding normal development and disease pathology. The Visium Spatial Gene Expression Solution allows for the investigation of spatially resolved whole transcriptome mRNA expression, while capturing histological information in the same tissue section. Using this solution, gene expression profiles can be mapped back to their original location, providing a new view of tissue and gene expression complexity as it applies to the study of cancer, immuno-oncology, neuroscience, developmental biology, and beyond. This method will be used for characterizing TME and tumor phenotype changes upon treatment with ICG-001 and ICG-001 and CAR T cells.

Combining CAR T therapy with the specific CBP/β-catenin antagonist ICG-001 should safely modify the tumor and TME towards differentiation/neurogenesis and stimulate endogenous immune responses. Based on the tumor heterogeneity defined in this aim, and the potential for antigen escape, these studies will investigate expression of other biomarkers within a panel of PBTs to explore potential combinational CAR T cell approaches to assess treatment efficacy (CD133, MDR1, EGFR, CD47).

Statistical analyses will be performed separately for each treatment regimen depending on the route of administration and model (EP or glioma). All statistical analyses will be done using SAS version 9.4 and Prism version 6. Tumor growth will be monitored by Xenogen bioluminescent imaging, and mean signal intensities will be obtained from scans at multiple time points for each mouse. Results will be analyzed using random intercept and slope regression models with intensities analyzed on a log scale. Models will include quadratic time, group, and their interactions. Significant estimated group differences from curves will be analyzed using t-tests. In addition, t-tests on raw data will be used to confirm earliest time point of difference. Kaplan-Meier curves will be created in SAS version 9.4 and median survival with 95% confidence intervals will be estimated. Survival curves obtained in Aim 1 will be compared using log-rank test. Pairwise comparisons will be made with Sidak for multiple comparisons.

Following demonstration of pre-clinical proof-of-concept, this combination approach could be rapidly translated to the clinic, as specific CBP/β-catenin antagonists, as single agents and in combination with chemotherapy, have been proven safe and efficacious in clinical trials.

Successful demonstration of preclinical proof-of-concept in this study could lead to rapid translation of this combination approach to the clinic to benefit patients with BT, because specific CBP/β-catenin antagonists, as single agents and in combination with chemotherapy, have been proven safe and efficacious in clinical trials. Furthermore, in preclinical studies, ICG-001 has demonstrated the ability to ameliorate chemotherapy-induced toxicity via beneficial effects on the normal somatic stem cell population (Teo et al. 2005; Manegold et al. 2018; Ma et al. 2005; Lukaszewicz et al. 2019).

### Example 4: Targeting WNT Signaling for Improved Glioma Immunotherapy

Despite aggressive standard-of-care therapy, including surgery, radiation, and chemotherapy, glioma recurrence is almost inevitable and uniformly lethal (Muldoon et al. 2007). In glioma, WNT pathway activation has been associated with a poor prognosis and progressive neurological deficits (Martin-Orozco et al. 2019). WNT signaling is associated with the proliferation of stem-like cells in human glioblastoma multiforme (GBM) (Kahlert et al. 2012a, Kahlert et al. 2015, Kahlert et al. 2012b) and resistance to chemotherapy, radiotherapy and immunotherapy (Sharma et al. 2021, Huang et al. 2020, Paw et al. 2015). β-catenin transcriptional activation, involving its translocation to the nucleus, is a hallmark of WNT pathway activation, and has been identified in 19% of adult and in 30% of pediatric gliomas.

Unbiased profiling studies demonstrated a strong negative correlation between cancer cell stemness and antitumor immunity signatures across 21 types of solid tumors, with reduced anticancer immune cell tumor infiltration, i.e. CD8+ T cells, natural killer cells and B cells, and increased tumor associated macrophages (Miranda et al. 2019). Similar, results were obtained from TCGA and tissue microarray analyses negatively correlating cancer stemness with tumor infiltrating activated CD4⁺ and CD8⁺ T cells (Malta et al. 2018). Cancer stem cells (CSC) also secrete various growth factors and cytokines to inhibit immune responses and promote an immunosuppressive tumor microenvironment (Chen et al. 2021) . Wnt/β-catenin signaling is frequently up-regulated in brain tumors and particularly in CSC (Wiese et al. 2020).

Furthermore, network expansion and the accumulation of Frizzled 1 (Fz1) in tumor microtubule (TM) projections might deleteriously influence neighboring neurons (Portela et al. 2019). TM, which enwrap and deplete Wingless (Wg) from neurons, while accumulating the Wnt receptor Fz1 could lead to activation of WNT pathway in glioma. Activation of WNT causes tumor cell proliferation, enhanced invasiveness via upregulation of JNK and accumulation of metalloproteases, with concomitant neuronal degeneration due to decreased required WNT maintenance (Portela et al. 2019, Kahn 2018, Kahn 2014). Glioma stem cells (GSCs), via secretion of the Wnt-induced signaling protein 1 (WISP1 ) can facilitate a ‘cold’ TME by promoting the survival of both GSCs and tumor-associated macrophages (TAMs) (Tao et al. 2020).

Wnt/β-catenin signaling and glioma resistance. Clinical and preclinical data suggest that curative immunotherapy must not only address immunotolerance and target tumor antigens, but also circumvent intrinsic as well as evolving barriers of adaptive and acquired escape mechanisms. A common mechanism of resistance involves activation of the WNT/β-catenin signaling pathway (Luke et al. 2019). Therefore, the role of aberrant WNT/b-catenin activation in immune exclusion and resistance to glioma immunotherapy was investigated. Increased expression of β-catenin in tumors has been correlated with a lack of intratumoral CD8+ T cells and the absence of dendritic cells (Spranger et al. 2015). The hostile tumor microenvironment (TME) leads to loss of therapeutic efficacy of immunotherapy, tumor antigen vaccination, and adoptive T cell transfer immunotherapy (including CAR T cell) approaches (Gajewski et al. 2017, Spranger et al. 2017, Horton et al. 2018, Osawa et al. 2019). Further support for the concept that increased WNT/β-catenin activity plays a role in immunotherapy response was provided by a patient, who had a complete initial response to CAR T cell therapy after presenting with a relatively high tumor mutational burden, T cell inflamed tumors, and low WNT/β-catenin activity, however upon relapse demonstrated increased intratumoral β-catenin/wnt (Brown et al. 2016). In the current study, it was demonstrated that the specific, small molecule CBP/β-catenin antagonist ICG-001 induces cytostasis and initiates differentiation of multiple glioma cell lines derived from patient (PDX) and murine tumors (GL261, K-Luc), and is synergistic when used in combination with CAR T cells. Downregulation of the WNT/CBP/β-catenin target gene survivin/BIRC5 with upregulation of the Wnt/p300/β-catenin target gene EphB2, after treatment with ICG-001 demonstrated selective inhibition of CBP/β-catenin signaling in glioma. The effect of ICG-001 in vitro on selected PBT030 and PBT147 cell lines was more broadly characterized using both Nanostring gene expression and proteomic analysis. These experiments demonstrated significant effects of ICG-001 treatment on tumor cell metabolism and immunopresentation. Furthermore, ICG-001 inhibition of the WNT/CBP/p-catenin signaling demonstrated enhanced CD3⁺, CD8⁺ and CD4⁺ cell infiltration with increased expression of the endothelial cell marker CD31, in a syngeneic mouse model of glioma (K-luc). Nanostring analysis of the K-luc syngeneic tumors showed that the top 20 up- and down-regulated genes were associated with tumor cell differentiation and endogenous immune cell activation. ICG-001 treatment of CAR T cells induced their differentiation into multiple types of effector and memory T cells (T cells derived from 3 donors were analyzed). ICG-001 enhanced the expression of genes involved in the cellular recruitment, stimulation of the immune synapse, adhesion, co-stimulation, cell survival and proliferation including DPP4, CD48, CEBPB, B2M, ICOSLG, CD81, CXCR4, XBP1, and NFIL3.

The refractory nature of gliomas provides a compelling motivation for developing novel treatment interventions, such as CAR T cell therapy, for glioma as well as other devastating malignancies (Brown et al. 2015, Brown et al. 2016, Garvin et al. 2012, Zhao et al. 2012, Khawraw et al. 2020). Taken together, these results demonstrate that inhibition of WNT/CBP/β-catenin signaling can induce glioma cell differentiation in vivo and in vitro and potentially, via modification of the TME, enhance immunotherapeutic interventions in glioma patients.

Materials and Methods

PBT tumor cells lines are patient derived tumors dissociated and grown in DMEM/F12 medium, supplemented with heparin, hepes, glutamax and B27. EFG and FGF is added at the time of the culture as described previously (Brown et al. 2009). ICG-001 was kindly provided by another laboratory.

In vitro experiments. Co-culture assay of PBT cell lines grown as described above and seeded to 100,000 cells/2 ml in 6 well plates. ICG-001 is added to the cell culture in concentrations 0, 5, 10 mM for 24-72 h, as described previously (Brown et al. 2013).

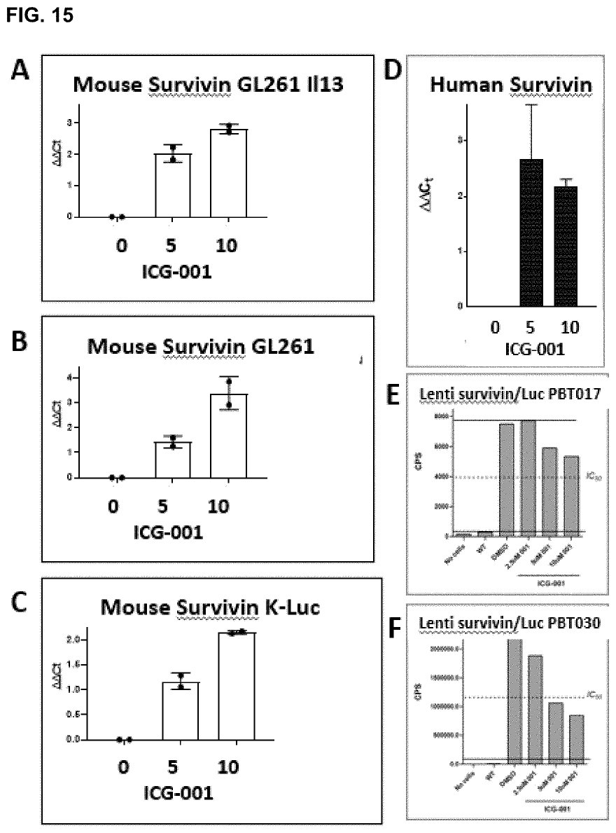

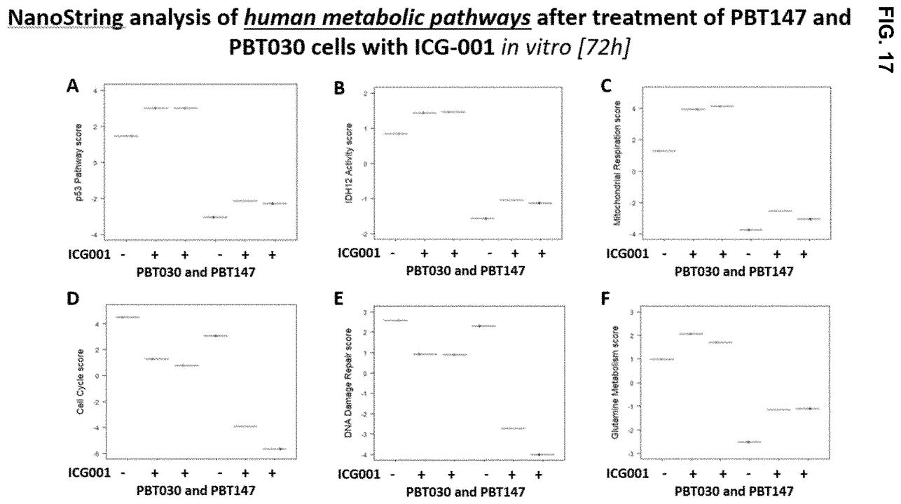

Proteomics data analysis. The normalized distributions of protein levels were found to be, as usual for such analyses, heavily skewed to the right. Therefore, a logarithmic transform of the data was worked with, which resulted in an approximate normal distribution of log protein levels. The densities of the log-transformed normalized protein level for each cell line/dose are shown in FIG. 15. To examine the effect of the drug, the difference in log normalized levels for each protein between the four treated samples and the appropriate untreated sample was calculated, which corresponds to examining the log of the fold changes in each protein after treatment. For each of the two cell lines, the log fold change for each protein at 5 μg was plotted against the log fold change at 10 μg. These plots are shown in FIG. 17, with the points colored-coded by cell line. The line of (linear) regression is calculated and shown for each cell line. This highlights a somewhat positive linear relationship between the log fold changes at 5 μg and 10 μg, with those proteins more upregulated at 5 μg being proportionately more so at 10 μg. However, the dose effect is significantly greater in PB030 than PB147, with the same increase in dose, resulting in a generally bigger fold change.

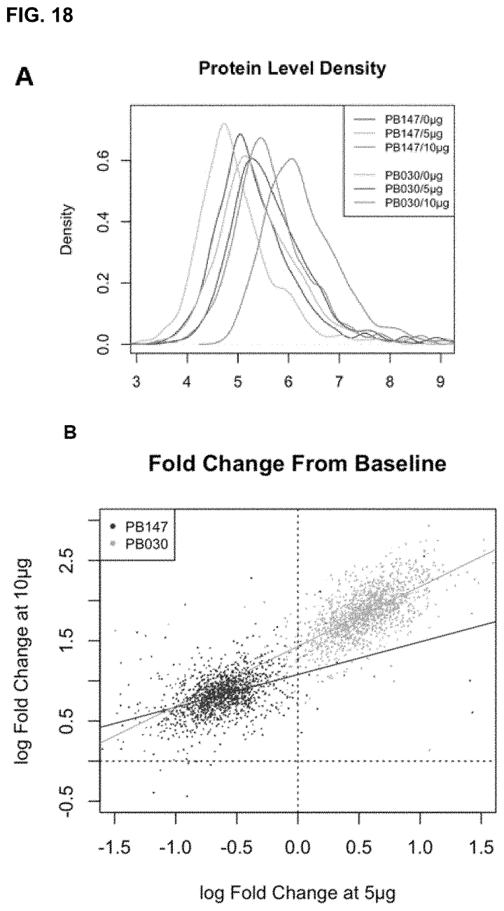

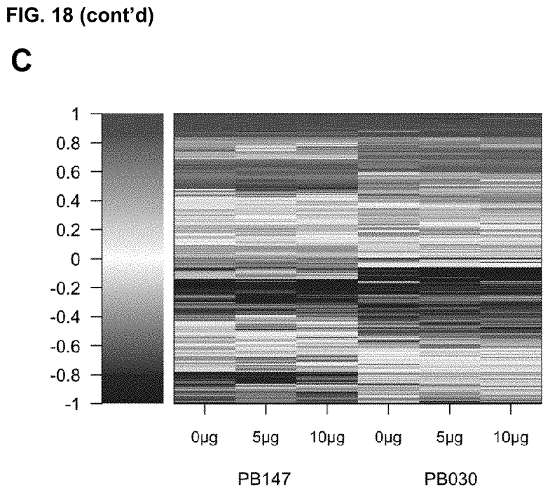

Patterns of similarities in protein regulation over cell lines and/or doses are explored in FIG. 18, which shows a heat map of up- or down-regulation of proteins. The heatmap has been constructed by using Ward's method for clustering and the Manhattan (L1) distance. To highlight patterns of similar changes in regulation over cell-lines and doses, rather than absolute differences in protein levels, quantile normalization has been used before creating the heatmap, with protein levels in each sample being normalized those of a uniform distribution on −1≤x≤1.

Animal studies. Syngeneic mice (C57BL/6) of 8-12 weeks of age were implanted with subcutaneous K-Luc tumors (n=8). 7 days after, when tumors became palpable and after confirmation of the tumor presence with BLU imaging, mice were implanted with Alzet minipumps with ICG001 (50 mg/kg/day). Pumps were continuously providing a daily dose of ICG-001. Tumor tissues were harvested on days 7, 14, 21 post pump implantation and tumors were prepared for IHC (paraffin sections) and Nanostring analysis.

IHC was performed in COH pathology core. Tissues were scanned and quantification of CD4, CD8 and CD31 expression was done using ImageJ software.

Image analysis. Images were processed and analyzed in the ImageJ (Schneider et al. 2012, also see Schneider, C. A., Rasband, W. S., Image J, U. S. National Institutes of Health, Bethesda, Md., USA, https://imagej.nih.gov/ij/, 1997-2018). Images were first processed using the color threshold function to identify the NSCs. Then a binary mask was generated for these identified cells. A watershed filter was then applied to this processed mask and the ‘Analyze Particles’ tool with a 4 pixel minimum size was employed to quantify the cells identified.

Results

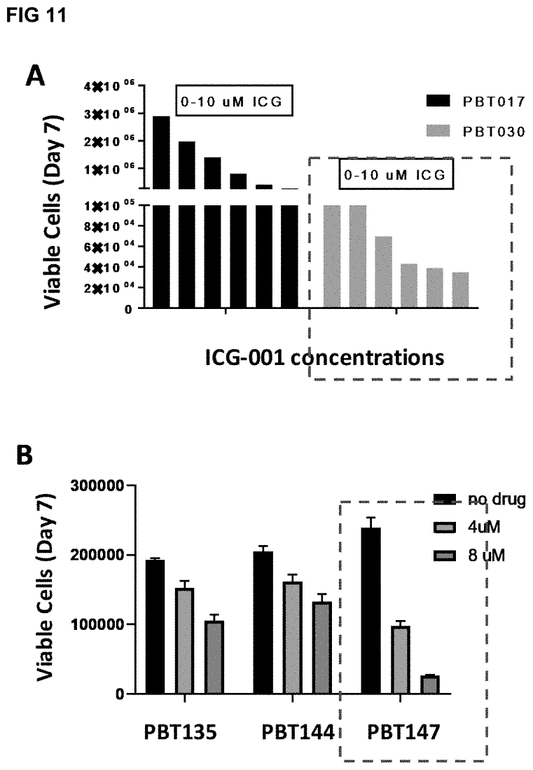

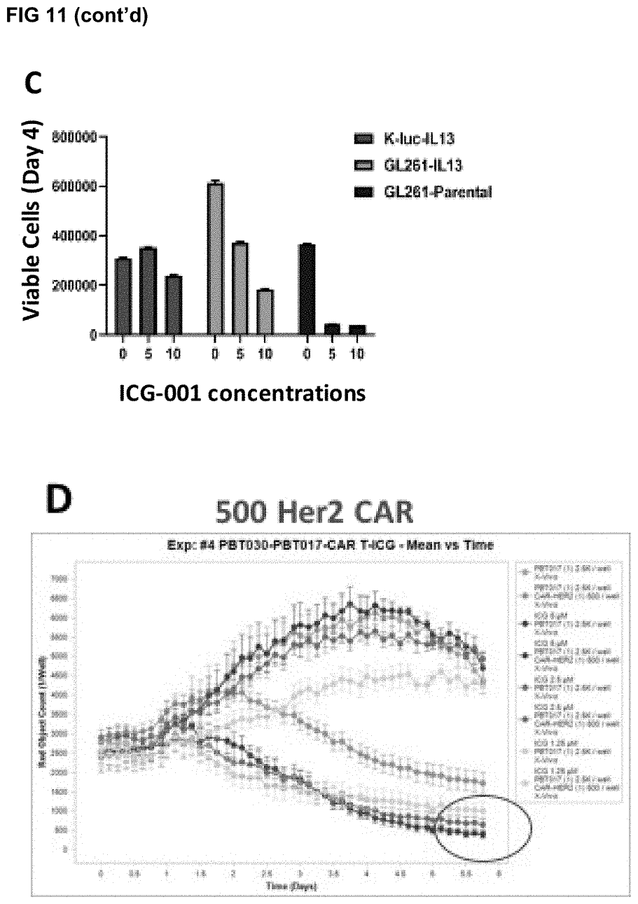

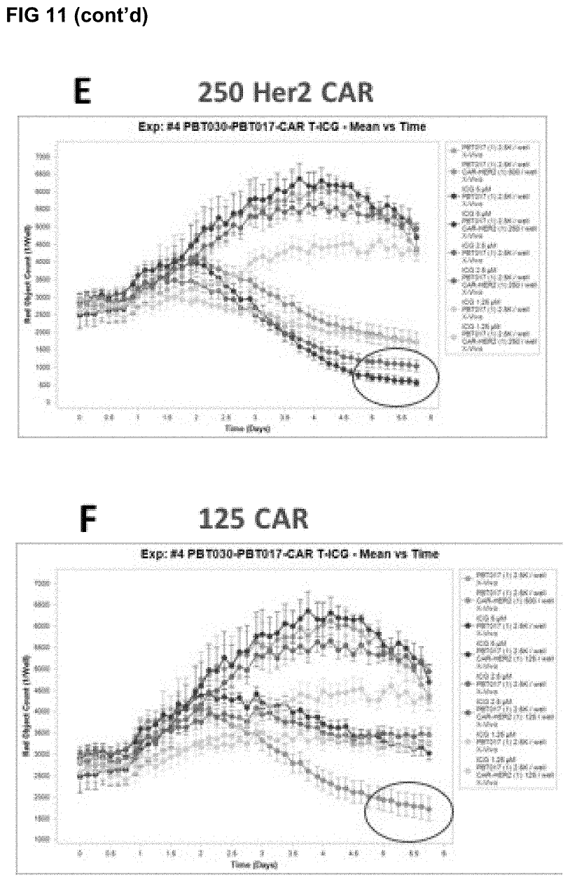

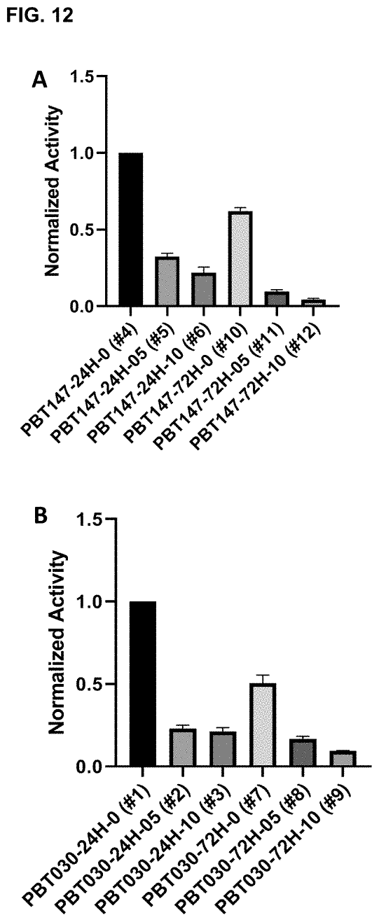

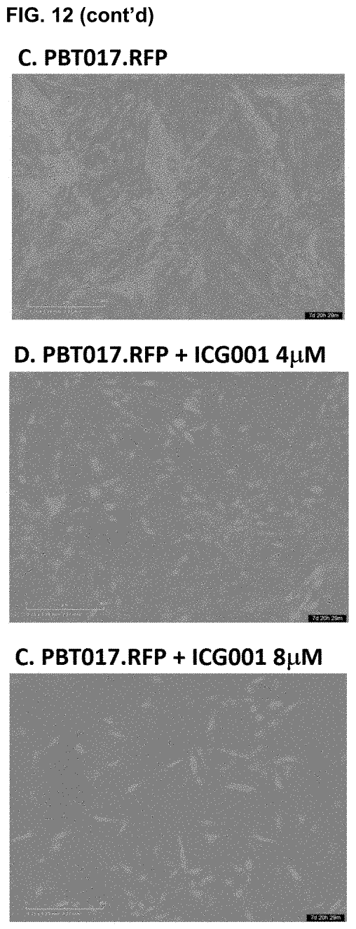

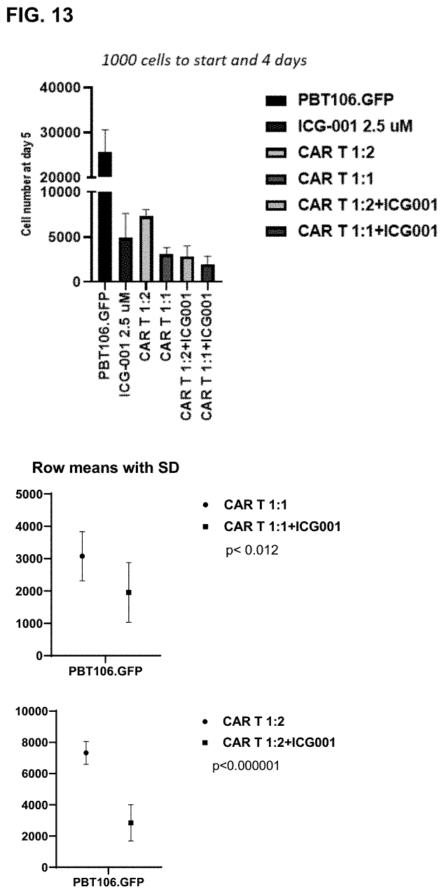

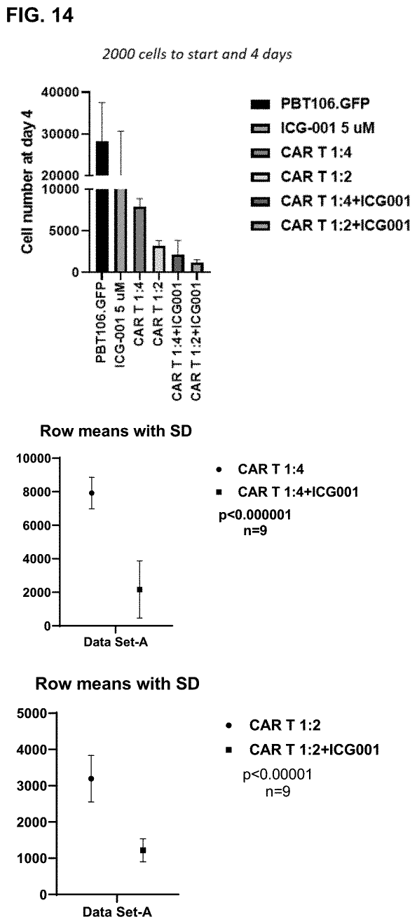

ICG-001 activity in vitro in human and mouse glioma cell lines. Initially, the effects of the specific CBP/β-catenin antagonist ICG-001 was tested in vitro as a single agent on human and mouse glioma cell lines (5 PBT human and 3 mouse glioma lines). Treatment with ICG-001 (0-10 μM) showed a concentration dependent cytostatic effect in all of the human patient-derived glioma (PBT017, PBT030, PBT135, PBT144, and PBT147) (FIG. 11A, B) and the three murine-derived glioma cell lines (K-luc, GL261-parental, and GL261.IL13Rα2 engineered) (FIG. 11C) tested. Furthermore, all glioma lines morphologically exhibited a less proliferative and more differentiated phenotype, loss of clonal expansion and downregulation of a key regulator of cell division KIF20A, when treated with ICG-001, as demonstrated with PBT017.RFP (FIG. 12) and improved killing by CAR T cells in Tumor[PBT106.eGFP]: CAR T [Her2] cells in ratios (1:2; 1:2, 1:4) (FIG. 13). This provided additional supporting data for using ICG-001 to stimulate glioma stem cell (GSC) differentiation and demonstrating that the effect of ICG-001 is not cell line dependent. It was also demonstrated that ICG-001-induced the differentiation of PBT147 and PBT030 cells in vitro by qRT-PCR with decreased expression of KIF20A, a critical regulator of controlling the mode of cell division during cortical neurogenesis and proliferation versus differentiation (FIG. 12[A,B]), (Geng et al. 2018).

Next, the efficacy of a titrated dose of CAR T cells±ICG-001 to induce tumor cell killing in vitro was tested (FIG. 11D-F). The combination of HER2-engineered CAR T (HER2-CAR T) cells and IGC-001 was evaluated using in vitro co-culture assays. ICG-001 in combination with HER2-CAR T cells demonstrated synergistic cytotoxicity in several patient in the PBT017 and PBT106 derived lines that express high levels of the tumor antigen HER2 (FIG. 11D, E, F and FIG. 13, 14). ICG-001 and HER2-CAR T cells in combination demonstrated synergistic killing in a CAR T cell concentration-dependent fashion (FIG. 11D-F). Enhanced killing was observed with combination of HER2-CAR T cells and ICG-001 at effector: target cell ratios of 1:5 and 1:10 (FIG. 11F).

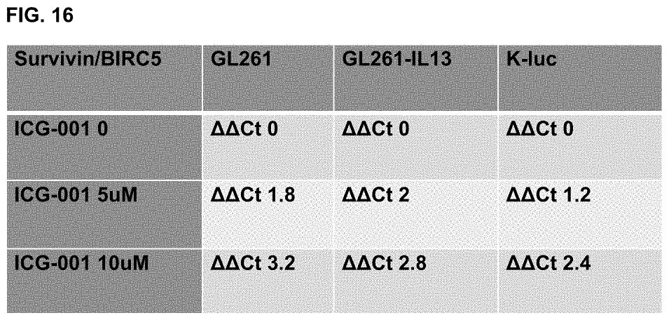

ICG-001 differentially affects the expression of the Wnt target genes BIRC5/Survivin and EphB2 in vitro. Using quantitative RT-PCR, it was demonstrated that ICG-001 selectively downregulated the expression of the Wnt/CBP/β-catenin target gene BIRC5/Survivin, while upregulating the expression of the Wnt/p300/β-catenin target gene EphB2, in both human and mouse glioma cell lines (Zhao et al. 2012). The effects of ICG-001 on Wnt/CBP/β-catenin signaling were further confirmed using a Survivin-luciferase reporter assay (FIG. 15). ICG-001's selective effects on the expression of Survivin and EphB2 may serve as indicators of ICG-001 on target tissue activity in vivo (FIG. 16) and a readout for pharmacokinetics/pharmacodynamics (PK/PD) as used previously in clinical trials with the second-generation CBP/β-catenin specific antagonist PRI-724 (Anthony et al. 2013).

ICG-001 Affects PBT tumor metabolism. The N-termini of the Kat3 coactivators, i.e. CBP and p300, provide a highly evolutionarily conserved hub to integrate multiple signaling cascades to coordinate cellular metabolism with the regulation of cellular status and function and specifically that p300/β-Catenin mediated transcription is required to enhance mitochondrial OXPHOS during the initiation of cellular differentiation (Hu et al. 2021, Thomas & Kahn 2016). Therefore, patient-derived GBM cell lines PBT147 and PBT030 lines were treated with ICG-001 (0, 5, or 10 μM) for 24 h or 72 h (FIG. 17 shown differences in both cells lines at 72 h). RNA isolated from the treated PBT cells was analyzed using the NanoString nCounter metabolic panel (NanoString Technologies). This approach digitally detects and counts gene transcripts in a single reaction without amplification (analysis done by City of Hope Molecular Pathology Core) (Geiss et al. 2008).

The results of these experiments indicated changes in several metabolic pathways, with a decrease in glycolysis and an increase in mitochondrial respiration, fully consistent with previous studies (Hu et al. 2021). This change in tumor cell metabolism may allow CD8 effectors the ability to more effectively compete for glucose within the TME (Bantug et al. 2018). Upregulation of glutamine metabolism, ROS, mTOR, and p53 signaling was also observed in the NanoString analysis (FIG. 17—data not shown). Importantly, antigen presentation was increased, with a concomitant decrease in immunosuppressive tryptophan/kynurenine metabolism (Vancurova et al. 2018) with treatment with ICG-001 at both time points (FIG. 17—data not shown). Downregulation of cell cycle-related genes was also observed, consistent with the previously described cytostatic effects and a switch from proliferation to differentiation, with the associated metabolic changes (Anthony et al. 2013). Fatty acid oxidation, the metabolic pathway preferred by “quiescent stem-like cells” (Knobloch et al. 2017), was decreased in all treated cell lines at all timepoints, indicating that ICG-001 was mediating GSC differentiation with metabolic reprogramming. Upregulation of the p53 tumor suppressive pathway and mitochondrial respiration, hallmarks of PBT cell differentiation, was observed at both time points and in both cell lines. Selected data is shown for PBT147 and PBT030 (FIG. 17).

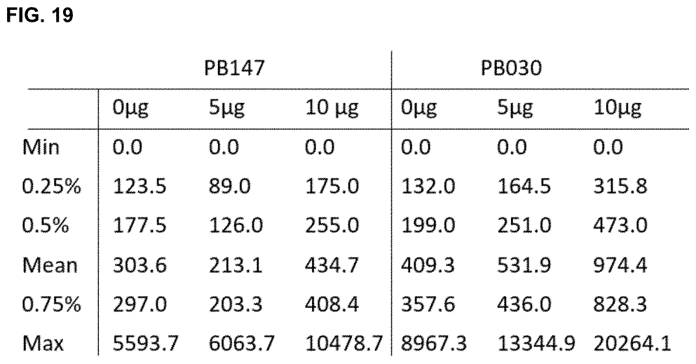

Proteomics analyses of the PBT147 and PBT030 glioma cell lines after treatment with ICG-001 to elucidate targeted pathways in vitro. In addition to the NanoString metabolic panel gene expression analysis of the glioma cell lines, a global proteomic analysis of PBT147 and PBT030 treated with ICG-001 (0, 5, and 10 μM) for 72 h (FIG. 18) was conducted. An overall increase in protein translation was observed with ICG-001 treatment that is associated with the activation of “quiescent stem-like cells”, metabolic reprogramming leading to increased ATP production, with an increase in protein synthesis and differentiation (FIGS. 18 and 19). Specifically, increased PKM1 and loss of HA and LDHA protein expression marks the transition from aerobic glycolysis to oxidative phosphorylation.

FIG. 19 shows the range of levels of the 1557 proteins that were measured in each sample at 72 h, after normalization. To account for differing numbers of cells in each sample, and to make numbers comparable across experiments, the levels were normalized by dividing the measured level for each protein by the total level of protein found in each sample.

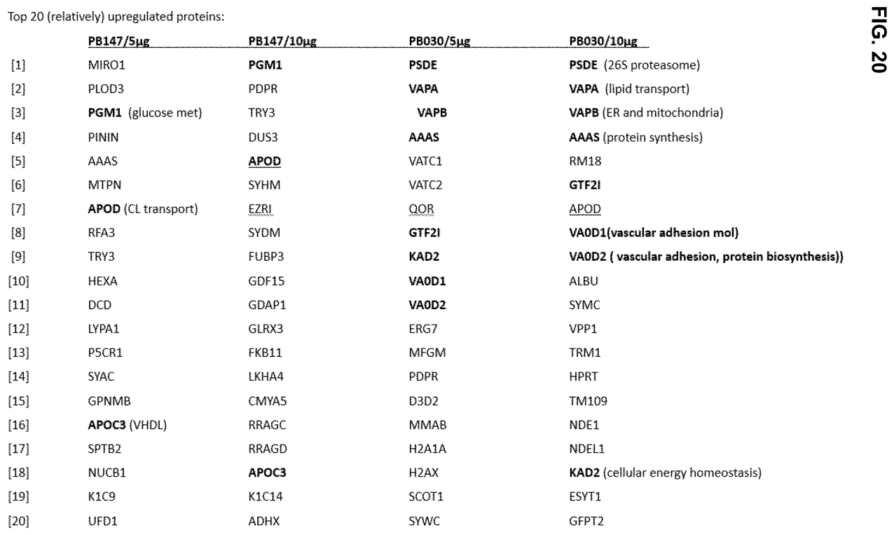

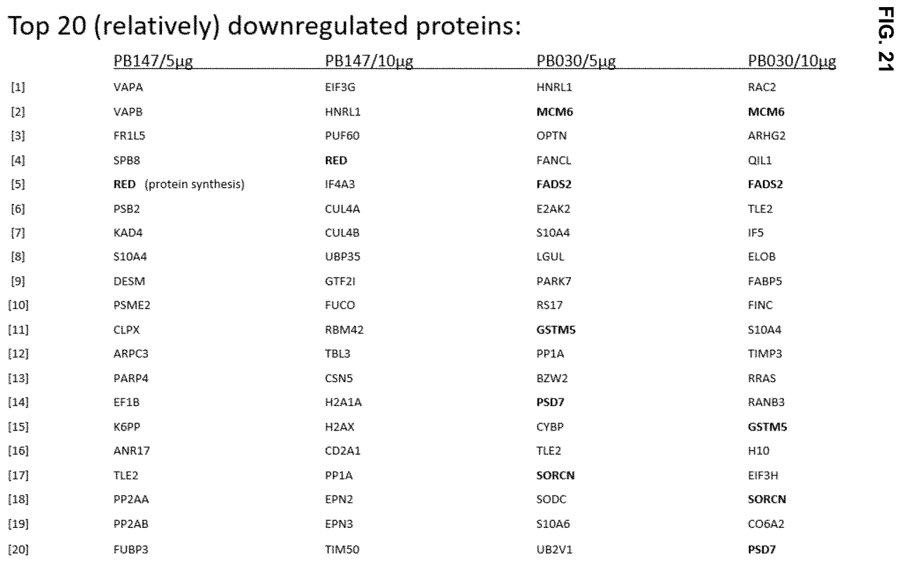

To examine the effect of ICG-001, the difference in log-normalized levels was calculated for each protein between the four treated samples and the appropriate untreated samples (which corresponds to looking at the log of the fold changes in each protein). For both cell lines, the log fold-change for each protein in the samples treated with 0, 5 and 10 pg of ICG001 was plotted against the log fold-change at 10 μg (see, e.g., FIG. 18, points color-coded by cell line). The line of (linear) regression is calculated and plotted for each cell line. This highlights a somewhat (positive) linear relationship between the log fold-changes at 5 μg and 10 μg of iCG-001 treated samples, with those proteins more upregulated at 5 pg being proportionately higher at 10 μg. However, the dose effect is significantly greater in PBT030 than PBT147 with the same increase in dose, resulting in a generally greater fold change. Patterns of similarities in protein regulation over cell lines and/or doses were also explored in FIG. 18, which shows a heat map of up- or downregulation of proteins. The heatmap has been constructed using Ward's method for clustering and the Manhattan (L1) distance. To highlight patterns of similar changes in regulation over cell lines and doses, rather than absolute differences in protein levels, quantile normalization before creating the heatmap has been used, with protein levels in each sample being normalized to those of a uniform distribution on −1≤x≤1 (FIGS. 18, 20-21).

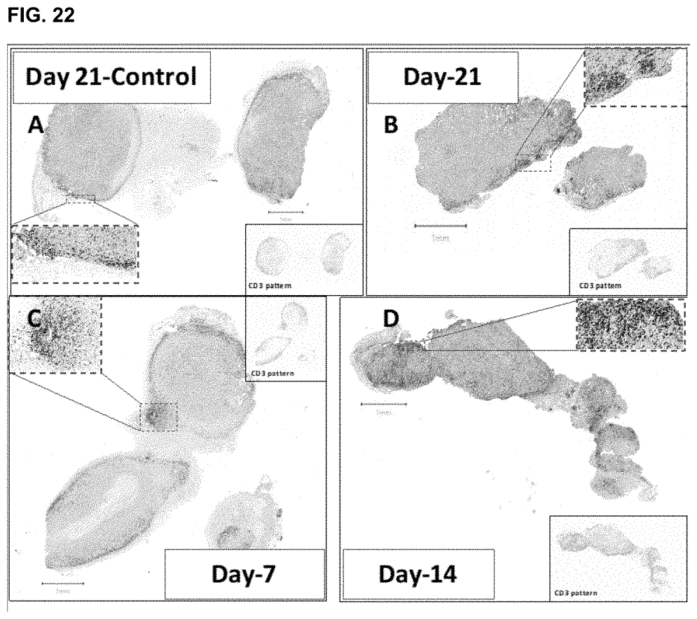

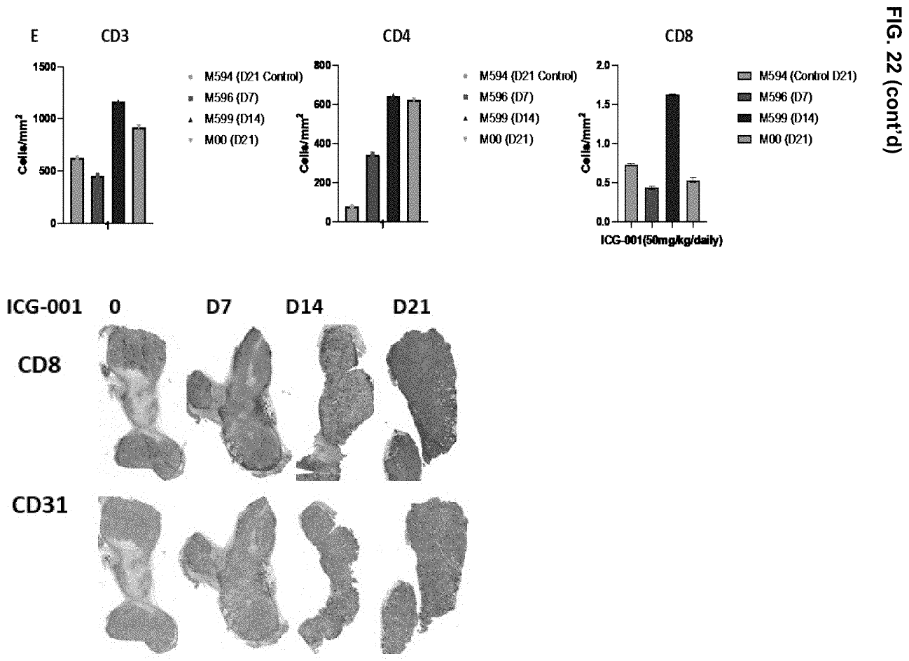

Antagonism of WNT/CBP/β-catenin signaling enhances infiltration of endogenous immune cells in syngeneic murine K-luc tumors Modulation of the “cold” TME should enhance the recruitment of T cells into the tumor (Duan et al. 2020). To test this, mice (C57BL/6) were implanted with subcutaneous K-luc tumors and treated with ICG-001 (delivered by subcutaneous Alzet minipumps, 50 mg/kg/day). The ICG-001-induced effects on endogenous immune response and reprogramming of the TME was evaluated on days 7, 14, and 21 post-pump implantations. An increase in mouse host immune cells including CD3⁺, CD8⁺, and CD4⁺ cells infiltrating the tumor was observed by day 14 post-ICG-001 treatment (FIG. 22). A time-dependent increase in CD31 expression was further observed (FIG. 22). These results support the notion that specific downregulation of WNT/CBP/β-catenin signaling with reprogramming the TME may enhance the efficacy of tumor immunotherapy with increased recruitment of host T-cells.

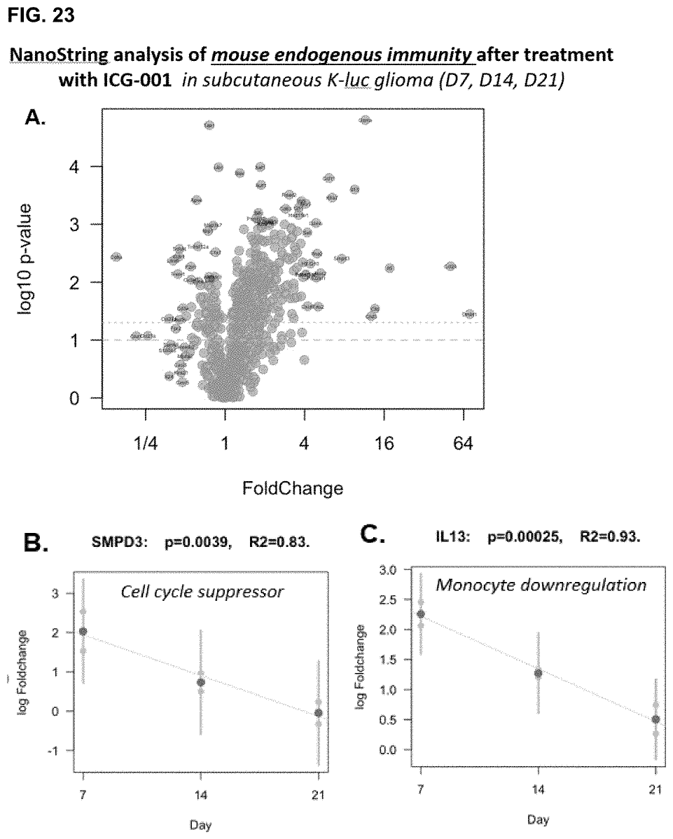

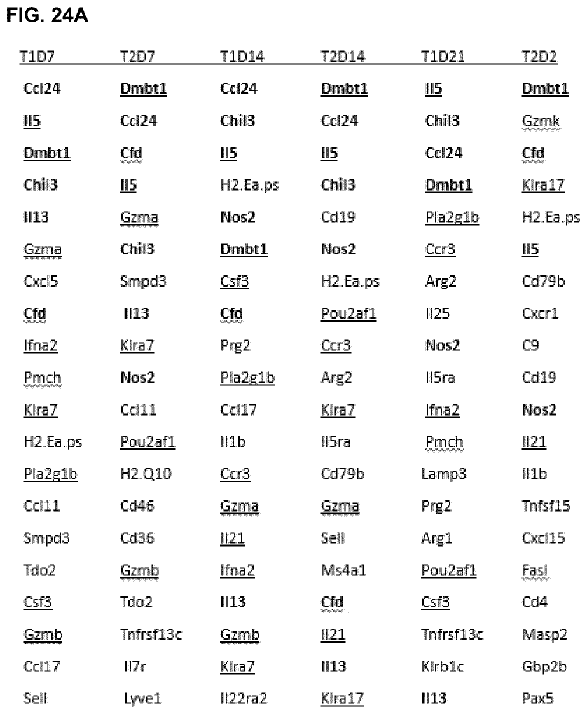

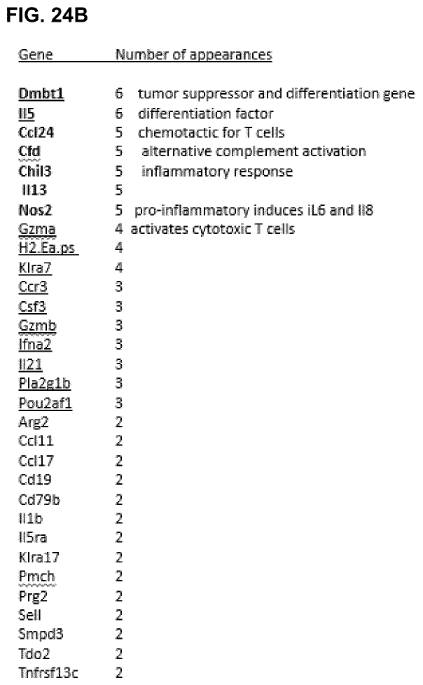

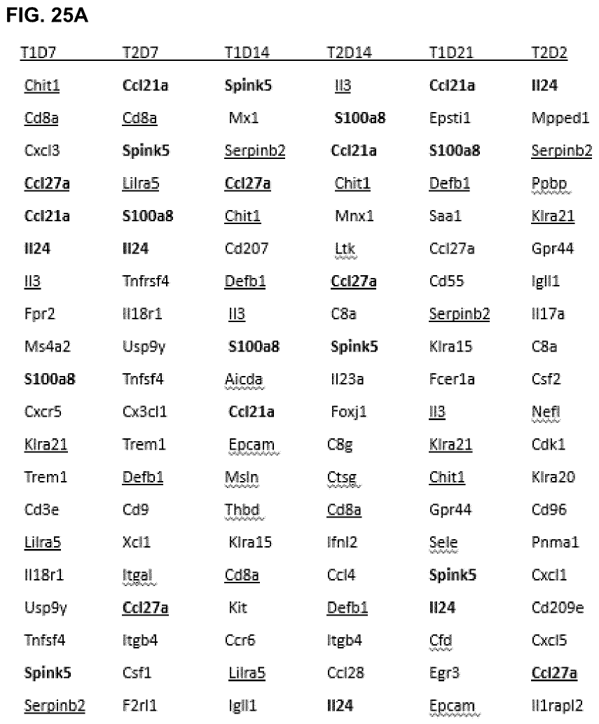

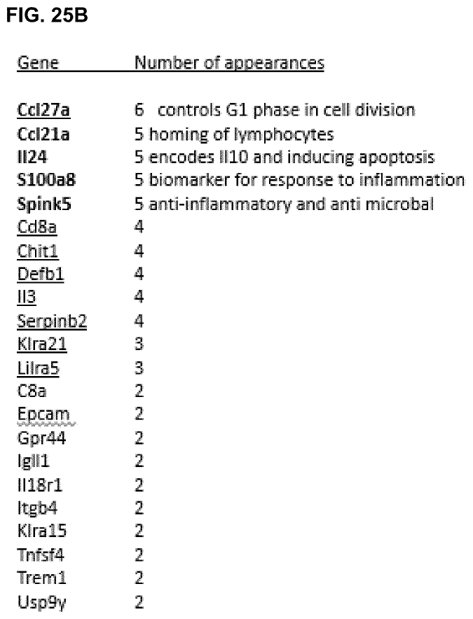

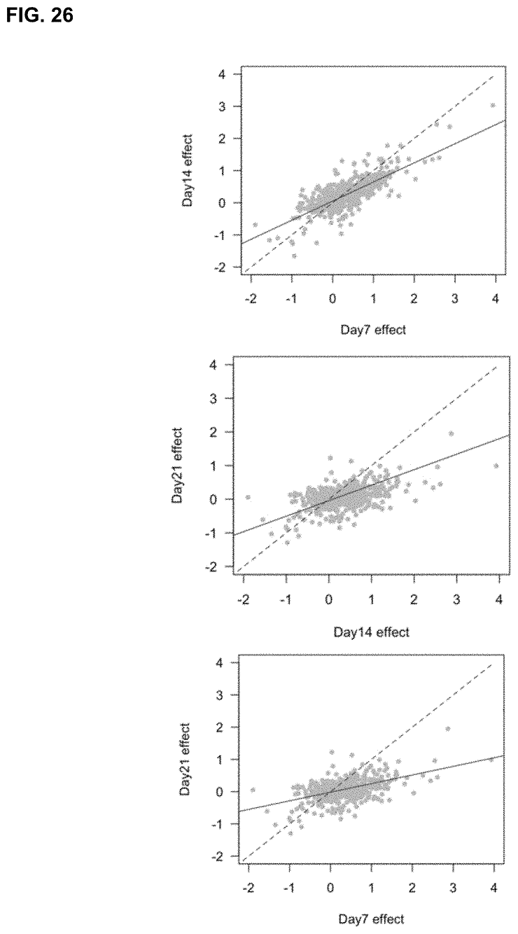

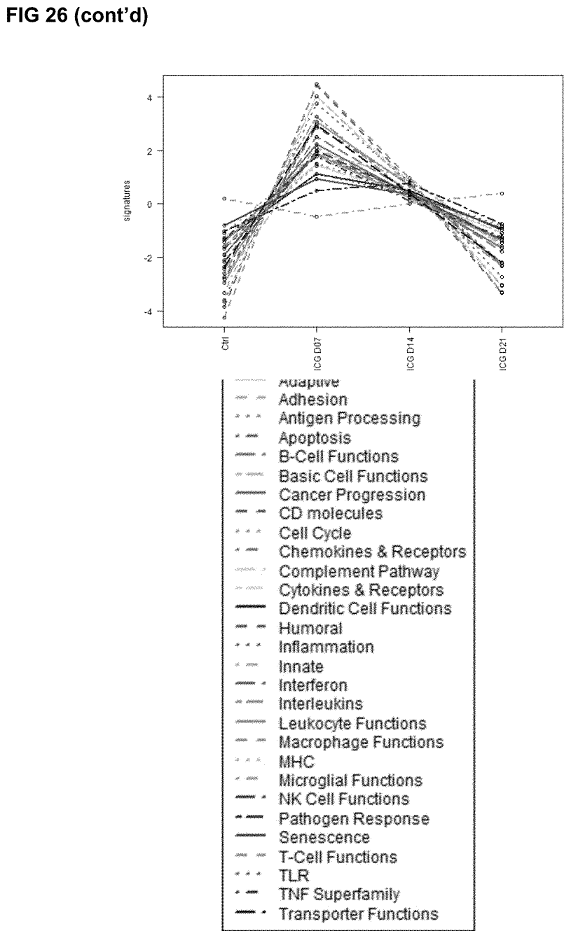

NanoString analysis of subcutaneous murine K-luc GBM tumors The ICG-001 and saline control treated tumors were harvested on days 7, 14, and 21. Freshly frozen K-luc tumors were used for RNA isolation and NanoString analysis to determine endogenous immune response upon treatment with ICG-001. An ICG-001 concentration dependent increase in immune pathways in mice treated with ICG-001 was observed when compared with untreated controls on day 21 after the start of ICG-001 administration. Essentially, the same upregulation/downregulation effects at each timepoint, except the amount of up/downregulation, diminished consistently with time in both tumors. Tumor 1 and tumor 2 responded very similarly, demonstrating a fold-change of each mRNA at day 7. The correlation continues through day 14 and day 21 but decreases likely due to the treatment effect in tumor 2 decreasing quicker than the treatment effect in tumor 1 (FIG. 23). The NanoString analysis data aligned with the IHC data above, suggesting the correct timing and ICG-001 dose for designing combinational therapies (FIGS. 23, 24, 25, 26).

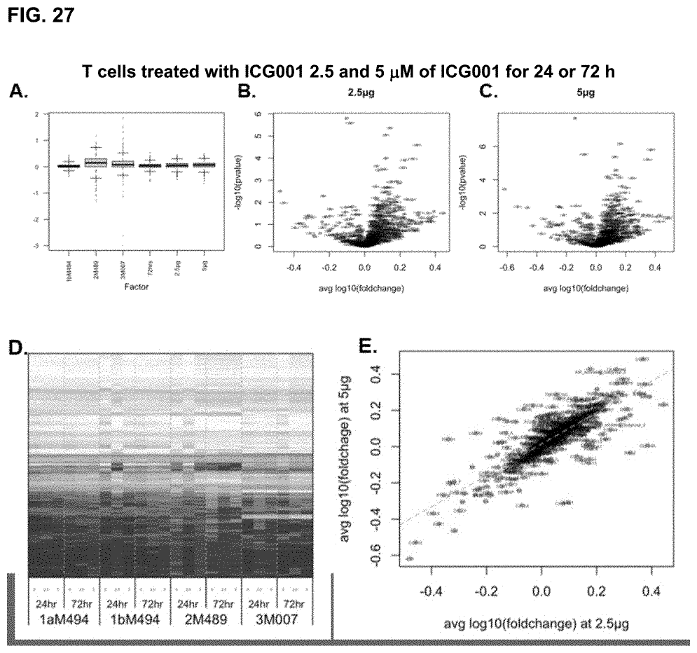

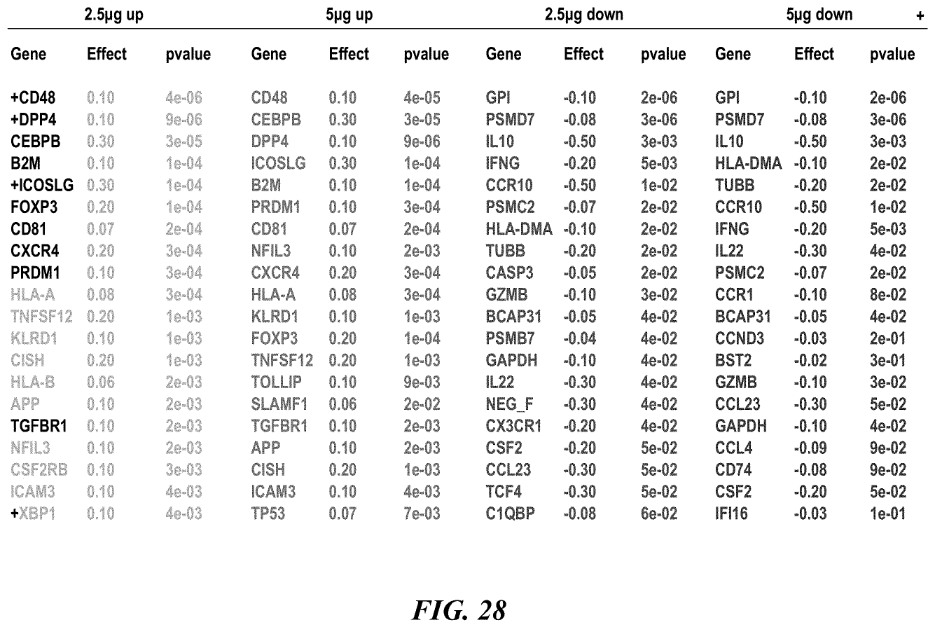

Antagonism of WNT/CBP/β-catenin signaling effects CAR T cells. Wnt signaling plays critical roles in T cell development and commitment (Li et al. 2019). Thus, the combined data derived from all T cell lines used and compared with untreated controls was analyzed for each T cell line. The analysis demonstrated that gene expression was not dependent on which T cell line was used and was not dependent on concentration of ICG-001 at 2.5 or 5 μM (FIGS. 27, 28).

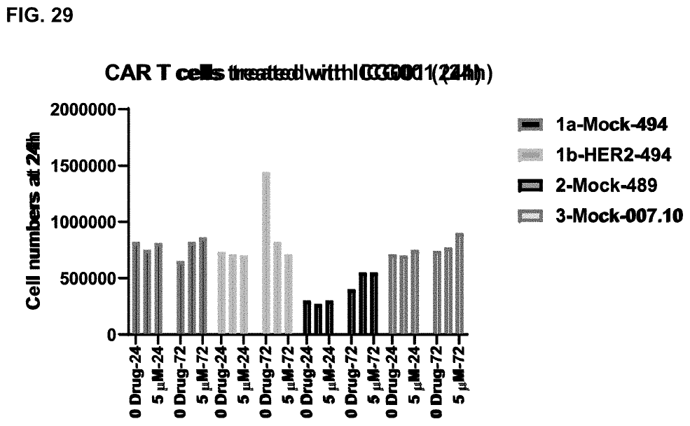

However, at a higher concentration of ICG-001, a higher degree of differential gene expression was observed. ICG-001 treatment directed T cell differentiation into multiple types of effector and memory T cells by activation of recruitment of signaling molecules and stimulation of immune synapse, adhesion, co-stimulation, cell survival, and proliferation. The immunologically active TME was supported by upregulation of key genes including DPP4, CD48, CEBPB, B2M, ICOSLG, CD81, CXCR4, XBP1, NFIL3, and others upon treatment with ICG-001 (FIG. 29). Note the function of those genes include the following:
DPP4—T cell activation, co-stimulation, Memory T cell generation
CD48—T-cell activation, recruitment of signaling molecules, immune synapse, adhesion, co-stimulation; regulation of effector/memory T cells generation and survival
CEBPB-myeloid cell activation, dendritic cell activation and Th17 priming
B2M-Immunologically active immune microenvironment
ICOSLG—a costimulatory signal for T-cell proliferation and cytokine secretion; induces also B-cell proliferation and differentiation into plasma cells
FOXP3-conversion of naïve T-cells to Treg cells
Cd81—to mediate signal transduction events, which are important for cells' development, activation, growth and motility
CXCR4—regulates T cell migration along gradients of the chemokine CXCL12, expressed on activated T cells
TGFBR1—active not suppressed autoimmunity
XBP1—IRE1a-XBP1 pathway activation regulating activation and differentiation of type-2 T helper cells (Th2)
NFIL3 attenuates the suppressive ability and stability of Treg cells

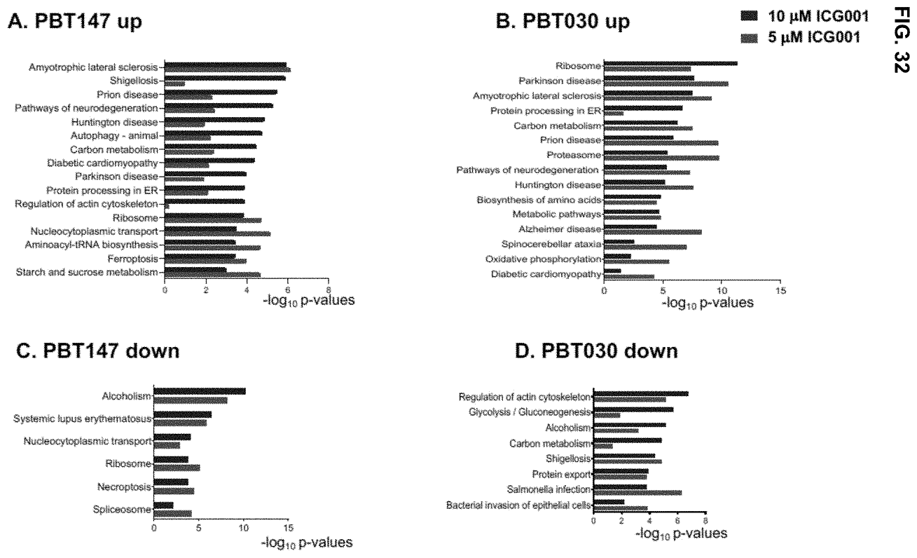

A pathway enrichment analysis was performed and shown in FIG. 32.

Discussion

Human models of GBM and CAR T-cell therapy have been widely published. Strengths of previous therapies include but are not limited to new CAR T cell design, improvements of pre-clinical BT models, and modulation of TME. However, previous studies have not taken the novel approach of using a non-toxic small molecule agent to differentiate tumor cells without deleterious effects on somatic stem cells³⁴, thus improving CAR T cell therapy. To ensure reproducibility of the data, a selective small molecule antagonist of the CBP/β-catenin interaction ICG-001 with CAR T cells was used in various human PBT models and in syngeneic mouse models (male and female mice).

One possible mechanism of activation of WNT in GBM tumor cells is via TM which enwraps neurons and depletes Wingless (Wg) from neurons, while accumulating the Wg receptor Fz1 on glioma cells and activation of WNT pathway. WISP1 plays important roles in maintaining GSCs and tumor-supportive TAMs in GBM, indicating that it may be targeting WNT/β-catenin-WISP1 signaling may effectively improve GBM treatment and patient survival (Tao et al. 2020). Using human glioma xenograft models with patient-derived tumors in NSG mice, it was found that IL13Bζ-CAR T cells improved anti-tumor activity and T cell persistence as compared to first-generation IL13-CAR CD8(+) T cells that had shown evidence for bioactivity in patients (Brown et al. 2018). These second generation IL13Bζ-CAR T cells will be used in future studies. However, these studies are not limited by the CAR and can be expanded to HER2-CAR therapies as well (Wang et al. 2020). To overcome known limitations of use of CAR T cell therapy for BTs, such as antigen escape and need for increased T cell persistence and potency, a novel combinational therapy approach with the WNT/β-catenin antagonist ICG-001 is proposed herein.

Establishing proof-of-concept that ICG-001 can safely target aberrant WNT/β-catenin signaling in BTs in combination with CAR T cell therapy could provide a novel way of broadening tumor response to CAR T cell therapy while decreasing resistance and relapse arising post-CAR T cell therapy in solid tumors. However, targeting WNT signaling with anything except a specific CBP/beta-catenin antagonist has associated toxicities (Kahn EOTT 2021). Therefore, the combination of a CBP/beta-catenin antagonist to safely differentiate away CSC with a CAR is novel. Separately, the immune subsets and WNT/β-catenin pathway activation in the TME of BTs in preclinical models was analyzed using novel techniques such as scRNA sequencing, NanoString and proteomics. To assess metabolic changes upon activation of the WNT pathway) and correlated these results with previously obtained data from patient samples obtained post-CART cell therapy (i.e., TCGA) (Aim 1), including outcome. CBP/β-catenin antagonists, by enhancing the immunostimulatory TME, may improve the response to immunotherapy by 1) eliminating CSCs via differentiation, 2) modifying the “cold” TME, and 3) changing the mode of division of CAR T cells. thereby, increasing the T-memory cell population and, thus, improving the outcome of patients treated with CAR T cell immunotherapy and expanding the population of patients with “cold” non-inflamed tumors.

A key gene of glioma cells differentiation, including ASCL1, has shown to contribute to the development of a glioma stem cell phenotype, which are thought to be the source of resistance and relapse after treatment (Azzarelli et al. 2022). Furthermore, reprogramming immune landscape with a single gene in non-cancer cells, but macrophages, identified the S100A4 as a regulator of an immune suppressive T and myeloid cell subtypes (Abdelfattah et al. 2022).

The outcome of these studies has demonstrated that ICG-001 induces positive differences in the gene expression of metabolic pathways, and that ICG-001 was not toxic to T cells in vitro promoting differentiation of both tumor and T cells, providing important insights for the advancement of CAR T cell therapy combined with ICG-001 for the treatment for BTs. These promising studies provide the basis for future development of this multi-targeted approach, designed to box-in tumor growth with the ultimate goal of developing a breakthrough treatment for patients with brain tumors.

### REFERENCES

All references cited below and in the disclosure are hereby incorporated by reference in their entirety, as if fully set forth herein.
Abdelfattah, N., et al. Single-cell analysis of human glioma and immune cells identifies S100A4 as an immunotherapy target. Nat Commun 13, 767 (2022).
Alizadeh, D., R. A. Wong, X. Yang, D. Wang, J. R. Pecoraro, C. F. Kuo, B. Aguilar, Y. Qi, D. K. Ann, R. Starr, R. Urak, X. Wang, S. J. Forman and C. E. Brown (2019). “IL15 Enhances CAR-T Cell Antitumor Activity by Reducing mTORC1 Activity and Preserving Their Stem Cell Memory Phenotype.” Cancer Immunol Res 7(5): 759-772.
Anthony B. El-Khoueiry , Y.N., Dongyun Yang , Sarah Cole , Michael Kahn , Marwan ZoghbiJennifer Berg , Masamoto Fujimori , Tetsuhi Inada , Hiroyuki Kouji , Heinz-Josef Lenz. A phase I first-in-human study of PRI-724 in patients (pts) with advanced solid tumors. Journal of Clinical Oncology 31(2013).
Azzarelli, R., et al. ASCL1 phosphorylation and ID2 upregulation are roadblocks to glioblastoma stem cell differentiation. Sci Rep 12, 2341 (2022).
Bantug, G. R., Galluzzi, L., Kroemer, G. & Hess, C. The spectrum of T cell metabolism in health and disease. Nat Rev Immunol 18, 19-34 (2018).
Bhaduri, A., E. Di Lullo, D. Jung, S. Muller, E. E. Crouch, C. S. Espinosa, T. Ozawa, B. Alvarado, J. Spatazza, C. R. Cadwell, G. Wilkins, D. Velmeshev, S. J. Liu, M. Malatesta, M. G. Andrews, M. A. Mostajo-Radji, E. J. Huang, T. J. Nowakowski, D. A. Lim, A. Diaz, D. R. Raleigh and A. R. Kriegstein (2020). “Outer Radial Glia-like Cancer Stem Cells Contribute to Heterogeneity of Glioblastoma.” Cell Stem Cell 26(1): 48-63 e46.
Brown, C. E., B. Aguilar, R. Starr, X. Yang, W. C. Chang, L. Weng, B. Chang, A. Sarkissian, A. Brito, J. F. Sanchez, J. R. Ostberg, M. D'Apuzzo, B. Badie, M. E. Barish and S. J. Forman (2018). “Optimization of IL13Ralpha2-Targeted Chimeric Antigen Receptor T Cells for Improved Anti-tumor Efficacy against Glioblastoma.” Mol Ther 26(1): 31-44.
Brown, C. E., D. Alizadeh, R. Starr, L. Weng, J. R. Wagner, A. Naranjo, J. R. Ostberg, M. S. Blanchard, J. Kilpatrick, J. Simpson, A. Kurien, S. J. Priceman, X. Wang, T. L. Harshbarger, M. D'Apuzzo, J. A. Ressler, M. C. Jensen, M. E. Barish, M. Chen, J. Portnow, S. J. Forman and B. Badie (2016). “Regression of Glioblastoma after Chimeric Antigen Receptor T-Cell Therapy.” N Engl J Med 375(26): 2561-2569.
Brown, C. E., B. Badie, M. E. Barish, L. Weng, J. R. Ostberg, W. C. Chang, A. Naranjo, R. Starr, J. Wagner, C. Wright, Y. Zhai, J. R. Bading, J. A. Ressler, J. Portnow, M. D'Apuzzo, S. J. Forman and M. C. Jensen (2015). “Bioactivity and Safety of IL13Ralpha2-Redirected Chimeric Antigen Receptor CD8+ T Cells in Patients with Recurrent Glioblastoma.” Clin Cancer Res 21(18): 4062-4072.
Brown, C. E. and C. L. Mackall (2019). “CAR T cell therapy: inroads to response and resistance.” Nat Rev Immunol 19(2): 73-74.
Brown, C. E., R. P. Vishwanath, B. Aguilar, R. Starr, J. Najbauer, K. S. Aboody and M. C. Jensen (2007). “Tumor-derived chemokine MCP-1/CCL2 is sufficient for mediating tumor tropism of adoptively transferred T cells.” J Immunol 179(5): 3332-3341.
Brown, C. E., et al. Recognition and killing of brain tumor stem-like initiating cells by CD8+ cytolytic T cells. Cancer Res 69, 8886-8893 (2009).
Brown, C. E., et al. Glioma IL13Ralpha2 is associated with mesenchymal signature gene expression and poor patient prognosis. PLoS One 8, e77769 (2013).
Chen, P., Hsu, W. H., Han, J., Xia, Y. & DePinho, R. A. Cancer Stemness Meets Immunity: From Mechanism to Therapy. Cell Rep 34, 108597 (2021).
Duan, Q., Zhang, H., Zheng, J. & Zhang, L. Turning Cold into Hot: Firing up the Tumor Microenvironment. Trends Cancer 6, 605-618 (2020).
Emami, K. H., C. Nguyen, H. Ma, D. H. Kim, K. W. Jeong, M. Eguchi, R. T. Moon, J. L. Teo, H. Y. Kim, S. H. Moon, J. R. Ha and M. Kahn (2004). “A small molecule inhibitor of beta-catenin/CREB-binding protein transcription [corrected].” Proc Natl Acad Sci USA 101(34): 12682-12687.
Gajewski, T. F., L. Corrales, J. Williams, B. Horton, A. Sivan and S. Spranger (2017). “Cancer Immunotherapy Targets Based on Understanding the T Cell-Inflamed Versus Non-T Cell-Inflamed Tumor Microenvironment.” Adv Exp Med Biol 1036: 19-31
Garvin, J. H., Jr., M. T. Selch, E. Holmes, M. S. Berger, J. L. Finlay, A. Flannery, J. W. Goldwein, R. J. Packer, L. B. Rorke-Adams, T. Shiminski-Maher, R. Sposto, P. Stanley, R. Tannous, I. F. Pollack and G. Children's Oncology (2012). “Phase II study of pre-irradiation chemotherapy for childhood intracranial ependymoma. Children's Cancer Group protocol 9942: a report from the Children's Oncology Group.” Pediatr Blood Cancer 59(7): 1183-1189.
Geiss, G. K., R. E. Bumgarner, B. Birditt, T. Dahl, N. Dowidar, D. L. Dunaway, H. P. Fell, S. Ferree, R. D. George, T. Grogan, J. J. James, M. Maysuria, J. D. Mitton, P. Oliveri, J. L. Osborn, T. Peng, A. L. Ratcliffe, P. J. Webster, E. H. Davidson, L. Hood and K. Dimitrov (2008). “Direct multiplexed measurement of gene expression with color-coded probe pairs.” Nat Biotechnol 26(3): 317-325.
Geng, A., et al. KIF20A/MKLP2 regulates the division modes of neural progenitor cells during cortical development. Nat Commun 9, 2707 (2018).
Holland, E. C. (2000). “Glioblastoma multiforme: the terminator.” Proc Natl Acad Sci USA 97(12): 6242-6244.
Horton, B. L., J. B. Williams, A. Cabanov, S. Spranger and T. F. Gajewski (2018). “Intratumoral CD8(+) T-cell Apoptosis Is a Major Component of T-cell Dysfunction and Impedes Antitumor Immunity.” Cancer Immunol Res 6(1): 14-24.
Hu, X., et al. Differential Kat3 Usage Orchestrates the Integration of Cellular Metabolism with Differentiation. Cancers (Basel) 13(2021).
Huang, M., D. Zhang, J. Y. Wu, K. Xing, E. Yeo, C. Li, L. Zhang, E. Holland, L. Yao, L. Qin, Z. A. Binder, D. M. O'Rourke, S. Brem, C. Koumenis, Y. Gong and Y. Fan (2020). “Wnt-mediated endothelial transformation into mesenchymal stem cell-like cells induces chemoresistance in glioblastoma.” Sci Transl Med 12(532).
Joshi, B. H., et al. Identification of interleukin-13 receptor alpha2 chain overexpression in situ in high-grade diffusely infiltrative pediatric brainstem glioma. Neuro Oncol 10, 265-274 (2008).
Kabir, T. F., C. A. Kunos, J. L. Villano and A. Chauhan (2020). “Immunotherapy for Medulloblastoma: Current Perspectives.” Immunotargets Ther 9: 57-77.
Kahn, M. Wnt Signaling in Stem Cells and Cancer Stem Cells: A Tale of Two Coactivators. Prog Mol Biol Transl Sci 153, 209-244 (2018).
Kahn, M. Can we safely target the WNT pathway? Nat Rev Drug Discov 13, 513-532 (2014).
Kahlert, U. D., D. Maciaczyk, S. Doostkam, B. A. Orr, B. Simons, T. Bogiel, T. Reithmeier, M. Prinz, J. Schubert, G. Niedermann, T. Brabletz, C. G. Eberhart, G. Nikkhah and J. Maciaczyk (2012a). “Activation of canonical WNT/beta-catenin signaling enhances in vitro motility of glioblastoma cells by activation of ZEB1 and other activators of epithelial-to-mesenchymal transition.” Cancer Lett 325(1): 42-53.
Kahlert, U. D., N. O. Bender, D. Maciaczyk, T. Bogiel, E. E. Bar, C. G. Eberhart, G. Nikkhah and J. Maciaczyk (2012b). “CD133/CD15 defines distinct cell subpopulations with differential in vitro clonogenic activity and stem cell-related gene expression profile in in vitro propagated glioblastoma multiforme-derived cell line with a PNET-like component.” Folia Neuropathol 50(4): 357-368.
Kahlert, U. D., A. K. Suwala, K. Koch, M. Natsumeda, B. A. Orr, M. Hayashi, J. Maciaczyk and C. G. Eberhart (2015). “Pharmacologic Wnt Inhibition Reduces Proliferation, Survival, and Clonogenicity of Glioblastoma Cells.” J Neuropathol Exp Neurol 74(9): 889-900.
Kahlon, K. S., C. Brown, L. J. Cooper, A. Raubitschek, S. J. Forman and M. C. Jensen (2004). “Specific recognition and killing of glioblastoma multiforme by interleukin β-zetakine redirected cytolytic T cells.” Cancer Res 64(24): 9160-9166.
Khasraw, M., D. A. Reardon, M. Weller and J. H. Sampson (2020). “PD-1 Inhibitors: Do they have a Future in the Treatment of Glioblastoma?” Clin Cancer Res.
Kim, J., A. Dey, A. Malhotra, J. Liu, S. I. Ahn, Y. J. Sei, A. M. Kenney, T. J. MacDonald and Y. Kim (2020). “Engineered biomimetic nanoparticle for dual targeting of the cancer stem-like cell population in sonic hedgehog medulloblastoma.” Proc Natl Acad Sci USA 117(39): 24205-24212.
Knobloch, M., et al. A Fatty Acid Oxidation-Dependent Metabolic Shift Regulates Adult Neural Stem Cell Activity. Cell Rep 20, 2144-2155 (2017).
Lai, K. K. Y., C. Nguyen, K. S. Lee, A. Lee, D. P. Lin, J. L. Teo and M. Kahn (2019). “Convergence of Canonical and Non-Canonical Wnt Signal: Differential Kat3 Coactivator Usage.” Curr Mol Pharmacol 12183.
Li, X., et al. WNT/beta-Catenin Signaling Pathway Regulating T Cell-Inflammation in the Tumor Microenvironment. Front Immunol 10, 2293 (2019).
Lukaszewicz, A. I., C. Nguyen, E. Melendez, D. P. Lin, J. L. Teo, K. K. Y. Lai, W. B. Huttner, S. H. Shi and M. Kahn (2019). “The Mode of Stem Cell Division Is Dependent on the Differential Interaction of beta-Catenin with the Kat3 Coactivators CBP or p300.” Cancers (Basel) 11(7).
Luke, J. J., R. Bao, R. F. Sweis, S. Spranger and T. F. Gajewski (2019). “WNT/beta-catenin Pathway Activation Correlates with Immune Exclusion across Human Cancers.” Clin Cancer Res 25(10): 3074-3083.
Ma, H., Nguyen, C., Lee, K. S. & Kahn, M. Differential roles for the coactivators CBP and p300 on TCF/beta-catenin-mediated survivin gene expression. Oncogene 24, 3619-3631 (2005).
Malta, T. M., et al. Machine Learning Identifies Stemness Features Associated with Oncogenic Dedifferentiation. Cell 173, 338-354 e315 (2018).
Manegold, P., K. K. Y. Lai, Y. Wu, J. L. Teo, H. J. Lenz, Y. S. Genyk, S. J. Pandol, K. Wu, D. P. Lin, Y. Chen, C. Nguyen, Y. Zhao and M. Kahn (2018). “Differentiation Therapy Targeting the beta-Catenin/CBP Interaction in Pancreatic Cancer.” Cancers (Basel) 10(4).
Marotte, L., S. Simon, V. Vignard, E. Dupre, M. Gantier, J. Cruard, J. B. Alberge, M. Hussong, C. Deleine, J. M. Heslan, J. Shaffer, T. Beauvais, J. Gaschet, E. Scotet, D. Fradin, A. Jarry, T. Nguyen and N. Labarriere (2020). “Increased antitumor efficacy of PD-1-deficient melanoma-specific human lymphocytes.” J Immunother Cancer 8(1).
Martin-Orozco, E., A. Sanchez-Fernandez, I. Ortiz-Parra and M. Ayala-San Nicolas (2019). “WNT Signaling in Tumors: The Way to Evade Drugs and Immunity.” Front Immunol 10: 2854.
Miranda, A., et al. Cancer stemness, intratumoral heterogeneity, and immune response across cancers. Proc Natl Acad Sci USA 116, 9020-9029 (2019).
Muldoon, L. L., C. Soussain, K. Jahnke, C. Johanson, T. Siegal, Q. R. Smith, W. A. Hall, K. Hynynen, P. D. Senter, D. M. Peereboom and E. A. Neuwelt (2007). “Chemotherapy delivery issues in central nervous system malignancy: a reality check.” J Clin Oncol 25(16): 2295-2305.
Neville, K. E., T. L. Bosse, M. Klekos, J. F. Mills, S. E. Weicksel, J. S. Waters and M. Tipping (2018). “A novel ex vivo method for measuring whole brain metabolism in model systems.” J Neurosci Methods 296: 32-43.
Okada, H., et al. Expression of glioma-associated antigens in pediatric brain stem and non-brain stem gliomas. J Neurooncol 88, 245-250 (2008).
Osawa, Y., E. Kojika, K. Nishikawa, M. Kimura, S. Osakaya, H. Miyauchi, T. Kanto, Y. Kawakami and K. Kimura (2019). “Programmed cell death ligand 1 (PD-L1) blockade attenuates metastatic colon cancer growth in cAMP-response element-binding protein (CREB)-binding protein (CBP)/beta-catenin inhibitor-treated livers.” Oncotarget 10(32): 3013-3026.
Paw, I., R. C. Carpenter, K. Watabe, W. Debinski and H. W. Lo (2015). “Mechanisms regulating glioma invasion.” Cancer Lett 362(1): 1-7.
Portela, M., V. Venkataramani, N. Fahey-Lozano, E. Seco, M. Losada-Perez, F. Winkler and S. Casas-Tinto (2019). “Glioblastoma cells vampirize WNT from neurons and trigger a JNK/MMP signaling loop that enhances glioblastoma progression and neurodegeneration.” PLoS Biol 17(12): e3000545.
Priceman, S. J., et al. Regional Delivery of Chimeric Antigen Receptor-Engineered T Cells Effectively Targets HER2(+) Breast Cancer Metastasis to the Brain. Clin Cancer Res 24, 95-105 (2018).
Ramakrishna, S., S. L. Highfill, Z. Walsh, S. M. Nguyen, H. Lei, J. F. Shern, H. Qin, I. L. Kraft, M. Stetler-Stevenson, C. M. Yuan, J. D. Hwang, Y. Feng, Z. Zhu, D. Dimitrov, N. N. Shah and T. J. Fry (2019). “Modulation of Target Antigen Density Improves CAR T-cell Functionality and Persistence.” Clin Cancer Res 25(17): 5329-5341.
Rusu, P., C. Shao, A. Neuerburg, A. A. Acikgoz, Y. Wu, P. Zou, P. Phapale, T. S. Shankar, K. Doring, S. Dettling, H. Korkel-Qu, G. Bekki, B. Costa, T. Guo, 0. Friesen, M. Schlotter, M. Heikenwalder, D. F. Tschaharganeh, B. Bukau, G. Kramer, P. Angel, C. Herold-Mende, B. Radlwimmer and H. K. Liu (2019). “GPD1 Specifically Marks Dormant Glioma Stem Cells with a Distinct Metabolic Profile.” Cell Stem Cell 25(2): 241-257 e248.
Sharma, P., et al. The Next Decade of Immune Checkpoint Therapy. Cancer Discov 11, 838-857 (2021).
Shergalis, A., A. Bankhead, 3rd, U. Luesakul, N. Muangsin and N. Neamati (2018). “Current Challenges and Opportunities in Treating Glioblastoma.” Pharmacol Rev 70(3): 412-445.
Schneider, C. A., Rasband, W. S. & Eliceiri, K. W. NIH Image to ImageJ: 25 years of image analysis. Nat Methods 9, 671-675 (2012).
Sommer, C., B. Boldajipour, T. C. Kuo, T. Bentley, J. Sutton, A. Chen, T. Geng, H. Dong, R. Galetto, J. Valton, T. Pertel, A. Juillerat, A. Gariboldi, E. Pascua, C. Brown, S. M. Chin, T. Sai, Y. Ni, P. Duchateau, J. Smith, A. Rajpal, T. Van Blarcom, J. Chaparro-Riggers and B. J. Sasu (2019). “Preclinical Evaluation of Allogeneic CAR T Cells Targeting BCMA for the Treatment of Multiple Myeloma.” Mol Ther 27(6): 1126-1138.
Song, K. S., et al. Long-term outcomes in children with glioblastoma. J Neurosurg Pediatr 6, 145-149 (2010).
Spranger, S., R. Bao and T. F. Gajewski (2015). “Melanoma-intrinsic beta-catenin signalling prevents anti-tumour immunity.” Nature 523(7559): 231-235.
Spranger, S., D. Dai, B. Horton and T. F. Gajewski (2017). “Tumor-Residing Batf3 Dendritic Cells Are Required for Effector T Cell Trafficking and Adoptive T Cell Therapy.” Cancer Cell 31(5): 711-723 e714.
Staal, F. J., T. C. Luis and M. M. Tiemessen (2008). “WNT signalling in the immune system: WNT is spreading its wings.” Nat Rev Immunol 8(8): 581-593.
Suwala, A. K., A. Hanaford, U. D. Kahlert and J. Maciaczyk (2016). “Clipping the Wings of Glioblastoma: Modulation of WNT as a Novel Therapeutic Strategy.” J Neuropathol Exp Neurol 75(5): 388-396.
Suwala, A. K., K. Koch, D. H. Rios, P. Aretz, C. Uhlmann, I. Ogorek, J. Felsberg, G. Reifenberger, K. Kohrer, R. Deenen, H. J. Steiger, U. D. Kahlert and J. Maciaczyk (2018). “Inhibition of Wnt/beta-catenin signaling downregulates expression of aldehyde dehydrogenase isoform 3A1 (ALDH3A1) to reduce resistance against temozolomide in glioblastoma in vitro.” Oncotarget 9(32): 22703-22716.
Tao, W., C. Chu, W. Zhou, Z. Huang, K. Zhai, X. Fang, Q. Huang, A. Zhang, X. Wang, X. Yu, H. Huang, Q. Wu, A. E. Sloan, J. S. Yu, X. Li, G. R. Stark, J. N. Rich and S. Bao (2020). “Dual Role of WISP1 in maintaining glioma stem cells and tumor-supportive macrophages in glioblastoma.” Nat Commun 11(1):3015.
Teo, J. L., Ma, H., Nguyen, C., Lam, C. & Kahn, M. Specific inhibition of CBP/beta-catenin interaction rescues defects in neuronal differentiation caused by a presenilin-1 mutation. Proc Natl Acad Sci U S A 102, 12171-12176 (2005).
Taylor, M. D., H. Poppleton, C. Fuller, X. Su, Y. Liu, P. Jensen, S. Magdaleno, J. Dalton, C. Calabrese, J. Board, T. Macdonald, J. Rutka, A. Guha, A. Gajjar, T. Curran and R. J. Gilbertson (2005). “Radial glia cells are candidate stem cells of ependymoma.” Cancer Cell 8(4): 323-335.
Thomas, P. D. and M. Kahn (2016). “Kat3 coactivators in somatic stem cells and cancer stem cells: biological roles, evolution, and pharmacologic manipulation.” Cell Biol Toxicol 32(1): 61-81.
Tran, F. H. and J. J. Zheng (2017). “Modulating the wnt signaling pathway with small molecules.” Protein Sci 26(4): 650-661.
Vancurova, I., Uddin, M. M., Zou, Y. & Vancura, A. Combination Therapies Targeting HDAC and IKK in Solid Tumors. Trends Pharmacol Sci 39, 295-306 (2018).
Wang, D., R. Starr, W. C. Chang, B. Aguilar, D. Alizadeh, S. L. Wright, X. Yang, A. Brito, A. Sarkissian, J. R. Ostberg, L. Li, Y. Shi, M. Gutova, K. Aboody, B. Badie, S. J. Forman, M. E. Barish and C. E. Brown (2020). “Chlorotoxin-directed CAR T cells for specific and effective targeting of glioblastoma.” Sci Transl Med 12(533).
Wang, M., Yin, B., Wang, H. Y. & Wang, R. F. Current advances in T-cell-based cancer immunotherapy. Immunotherapy 6, 1265-1278 (2014).
Wang, X., C. Berger, C. W. Wong, S. J. Forman, S. R. Riddell and M. C. Jensen (2011). “Engraftment of human central memory-derived effector CD8+ T cells in immunodeficient mice.” Blood 117(6): 1888-1898.
Wiese, M., et al. Combined treatment with CBP and BET inhibitors reverses inadvertent activation of detrimental super enhancer programs in DIPG cells. Cell Death Dis 11, 673 (2020).
Youngblood, B., J. S. Hale, H. T. Kissick, E. Ahn, X. Xu, A. Wieland, K. Araki, E. E. West, H. E. Ghoneim, Y. Fan, P. Dogra, C. W. Davis, B. T. Konieczny, R. Antia, X. Cheng and R. Ahmed (2017). “Effector CD8 T cells dedifferentiate into long-lived memory cells.” Nature 552(7685): 404-409.
Zah, E., E. Nam, V. Bhuvan, U. Tran, B. Y. Ji, S. B. Gosliner, X. Wang, C. E. Brown and Y. Y. Chen (2020). “Systematically optimized BCMA/CS1 bispecific CAR-T cells robustly control heterogeneous multiple myeloma.” Nat Commun 11(1): 2283.
Zhang, X., et al. Targeting role of glioma stem cells for glioblastoma multiforme. Curr Med Chem 20, 1974-1984 (2013).
Zhang, Z., C. Jiang, Z. Liu, M. Yang, X. Tang, Y. Wang, M. Zheng, J. Huang, K. Zhong, S. Zhao, M. Tang, T. Zhou, H. Yang, G. Guo, L. Zhou, J. Xu and A. Tong (2020). “B7-H3-Targeted CAR-T Cells Exhibit Potent Antitumor Effects on Hematologic and Solid Tumors.” Mol Ther Oncolytics 17: 180-189.
Zhao, X., Z. Liu, L. Yu, Y. Zhang, P. Baxter, H. Voicu, S. Gurusiddappa, J. Luan, J. M. Su, H. C. Leung and X. N. Li (2012). “Global gene expression profiling confirms the molecular fidelity of primary tumor-based orthotopic xenograft mouse models of medulloblastoma.” Neuro Oncol 14(5): 574-583.
Zhao, Y., D. Masiello, M. McMillian, C. Nguyen, Y. Wu, E. Melendez, G. Smbatyan, A. Kida, Y. He, J. L. Teo and M. Kahn (2016). “CBP/catenin antagonist safely eliminates drug-resistant leukemia-initiating cells.” Oncogene 35(28): 3705-3717.

I/we claim:

1. A method for treating a brain tumor comprising administering a population of cells expressing a chimeric antigen receptor that targets IL13Rα2 and/or HER2 in combination with a Wnt/β-catenin pathway modulator to a subject having a brain tumor

2. The method of claim 1, wherein the population of cells is administered before the Wnt/β-catenin pathway modulator, after the Wnt/β-catenin pathway modulator, or approximately simultaneously with the Wnt/β-catenin pathway modulator.

3. The method of claim 1, wherein the Wnt/β-catenin pathway modulator is a CPB/β-catenin antagonist.

4. The method of claim 3, wherein the CPB/β-catenin antagonist is ICG-001 or PRI-724.

5. The method of claim 1, wherein the brain tumor is an adult or pediatric brain tumor, and is a glioma, glioblastoma (GBM), medulloblastoma (MB), ependymoma, oligodendroglioma, or astrocytoma.

6. A population of cells expressing a chimeric antigen receptor that targets IL13Rα2 and/or HER2 for use in the treatment of a brain tumor, wherein the cells are administered before, during, or at approximately the same time as a Wnt/β-catenin pathway modulator.

7. The population of cells of claim 6, wherein the Wnt/β-catenin pathway modulator is a CPB/β-catenin antagonist.

8. The population of cells of claim 7, wherein the CPB/β-catenin antagonist is ICG-001 or PRI-724.

9. The population of cells of claim 6, wherein the brain tumor is an adult or pediatric brain tumor, and is a glioma, glioblastoma (GBM), medulloblastoma (MB), ependymoma, oligodendroglioma, or astrocytoma.
